(19) United States
(12) Patent Application Publication
(10) Pub. No.: US 2011/0059174 A1
(43) Pub. Date: Mar. 10, 2011

(54) PKCdelta REGULATES NEUROINFLAMMATORY EVENTS

(75) Inventors: ANUMANTHA GOUNDER KANTHASAMY, AMES, IA (US); ARTHI KANTHASAMY, AMES, IA (US)

(73) Assignee: IOWA STATE UNIVERSITY RESEARCH FOUNDATION, INC., AMES, IA (US)

(21) Appl. No.: 12/876,666

(22) Filed: Sep. 7, 2010

Related U.S. Application Data
(60) Provisional application No. 61/240,906, filed on Sep. 9, 2009.

(51) Int. Cl. A61K 9/10; A61K 38/20; A61K 38/19; A61K 38/48; A61K 38/46; A61K 33/40; A61K 33/00; A61K 31/7088; A61K 31/715; A61P 25/00; A61K 38/02

(52) U.S. Cl. 424/484; 424/85.2; 424/85.1; 424/94.63; 424/94.67; 424/616; 424/613; 424/718; 514/44 R; 514/54; 514/17.7

(57) ABSTRACT

This invention relates to methods and pharmaceutical compositions for regulating the levels of proinflammatory substances released from activated microglia using a protein kinase C delta (PKCd) inhibitor. In particular, it relates to decreasing the levels of proinflammatory substances produced within the microglial cells or released from activated microglical cells or both. Accordingly, the invention provides for the treatment of diseases, disorders, and conditions where neuroinflammation is implicated. The invention also relates to methods of using PKCd inhibitors and pharmaceutical compositions to treat diseases, disorders, and conditions associated with activated microglia.

### CROSS-REFERENCE TO RELATED APPLICATIONS

This application claims priority under 35 U.S.C. §119 of a provisional application Ser. No. 61/240,906 filed Sep. 9, 2009, and which application is hereby incorporated by reference in its entirety.

### GRANT REFERENCE

This invention was made with government support under NIH Grants NS038644, NS054016 and NS065167 awarded by the National Institutes of Health. The government has certain rights in the invention.

### BACKGROUND OF THE INVENTION

Inflammatory responses are mediated by immune defense cells that accumulate at the site of tissue injury or trauma to rid the body of unwanted exogenous agents (e.g., microbes) or endogenous agents (e.g., cancer cell clones); to clean up cellular debris, and to participate in tissue and wound healing. Unfortunately, the molecular mechanisms involved in these reparatory (inflammatory) processes can initiate secondary tissue damage, which, in turn, contributes to the pathogenesis and persistent pathology of several inflammatory diseases.

The microglial cell is the primary immunocompetent cell in the central nervous system. (Gehrmann et al., Brain Res. Rev., 20: 269 87, 1995; Giulian, D., J. Neurosci. Res., 18: 155 171, 1987; and Giulian et al., J. Neurosci., 15: 7712 26, 1995b). Microglia and infiltrating macrophages, produce and secrete of a number of pro-inflammatory cytokines and neurotoxic and other cytotoxic factors, for example, chemokines, proteases, reactive oxygen species, nitric oxide (NO) and other free radical species (Hartung et al., J. Neuroimmunol., 40:197-210, 1992; and Banati et al., Glia 7:111-8, 1993; and Ali et al., Adv. Rheumatol., 81:1-28, 1997). (Giulian et al., J. Neurosci., 9:4416-29, 1989; Giulian et al., Ann. Neurol., 27:33-42, 1990; Gehrmann et al., Brain Res. Rev., 20:269-87, 1995; Sobel, R. A., Neurol. Clin., 13:1-21, 1995; Dickson et al., Glia 7: 75-83, 1993; Benveniste, E. N., Res. Publ. Assoc. Res. Nerv. Ment. Dis., 72: 71-88, 1994; Sippy et al., J. Acquir. Defic. Syndr. Hum. Retrovirol., 10: 511-21, 1995; and Giulian et al., Neurochem, Int., 27:119-37, 1995a). Often these levels are increased in activated microglial cells or macrophages.

When microglia are activated they induce the synthesis and secretion of proteolytic enzymes, for example, proteinases that are implicated in the degradation of myelin and proteolysis of extracellular matrix proteins (Hartung et al., J. Neuroimmunol., 40:197-210, 1992; and Romanic et al., Brain Pathol., 4: 145-46, 1994). Such proteases include; cathepsins B, L, and S, the matrix metalloproteinases MMP-1, MMP-2, MMP-3, and MMP-9, and the metalloprotease-disintegrin ADAM8 plasminogen which forms outside microglia and degrades the extracellular matrix. Cathepsin B, MMP-1 and MMP-3 have been found to be increased in Alzheimer's disease (AD) and cathepsin B is increased in multiple sclerosis (MS).

In response to interferon-gamma (IFN-gamma) and TNF-alpha microglia have been found to release reactive oxygen intermediates that may cause tissue damage or even death of cells in the CNS. Microglia produce and secrete the cytokine interleukin 1 (IL-1), tumor necrosis factor alpha (TNF-alpha.). (Gehrmann et al., Brain Res. Rev., 20:269-87, 1995). In vitro studies have demonstrated that TNF-alpha is directly cytotoxic to oligodendrocytes and stimulates microglial phagocytosis of myelin (Zajicek et al., Brain 115:1611-31, 1992; and Soliven and Szuchet, Int. J. Dev. Neurosci., 13:351-67, 1995). In addition, TNF-alpha has been implicated in the pathogenesis of experimental autoimmune encephalomyelitis (EAE) and several other demyelinating diseases (Selmaj et al., J. Neuroimmunol., 56:135-41, 1995; Renno et al., J. Immunol., 154:944-53, 1995; Redford et al., Brain, 118:869-78, 1995; Probert et al., Proc. Natl. Acad. Sci. USA, 92:11294-8, 1995; and Probert et al., J. Leukoc. Biol., 59:518-25, 1996). Increased TNF-alpha. IL-1beta and IL-6 are observed in the substantia nigra and cerebral spinal fluids of Parkinson's disease patients (Hunot S, Hartmann A, Hirsh EC (2001). The inflammatory response in the Parkinson brain. Clin Neurosci Res 1:434-443; Nagatsu T, Mogi M, Ichinose H, Togari A (2000). Cytokines in Parkinson's disease. J Neural Transm (suppl) 58:143-151; Nagatsu T, Sawada M (2006). Cellular and molecular mechanisms of Parkinson's disease: neurotoxins, causative genes, and inflammatory cytokines. Cell Mol Neurobiol 26:779-800).

Microglia can express chemokines such as monocyte chemoattractant protein-1 (MCP-1) and others that induce the migration of inflammatory cells. Inflammatory cytokines like IL-10 and TNF-α, as well as bacterial-derived lipopolysaccharide (LPS) may stimulate microglia to produce MCP-1, MIP-1α, and MIP-1β. Cytokines and chemokines regulate most functions of mononuclear phagocytes (MNPs; monocytes), including the release of neurotoxic and cytotoxic factors.

Activated microglical cells release of proteases, inflammatory cytokines (including IL-1-beta, IL-6 and TNF-alpha.), chemokines, and neurotoxins. (Banati et al., Glia 7:111-118 (1993); Banati et al., Glia 7:183-191 (1993); Colton et al., FEBS Lett. 223:284-288 (1987); Dickson et al., Glia 7: 75-83 (1993). Microglial cells can be activated by any number of stimuli, for example, bacteria or viruses when they invade the CNS. Lipopolysaccharide (LPS), a component of the wall of gram-negative bacteria, is a known microglial activator. They have been implicated in the pathology of inflammatory joint diseases including rheumatoid arthritis (Rathanaswami et al., J. Biol. Chem. 268:5834-9, 1993; Badolato and Oppenhiem, Semin. Arthritis Rheum., 2:526-38, 1996; De Benedetti et al., Curr. Opin. Rheumatol., 9:428-33, 1997; Viliger et al., J. Immunol., 149:722-27, 1992; Hosaka et al., Clin. Exp Immunol., 97:451-7, 1994; Kunkel et al., J. Leukoc. Biol., 59:6-12, 1996). The release of inflammatory mediators including reactive oxygen prominent stimulator of microglia activation. Acute CNS insult, as well as chronic conditions such as HW encephalopathy, epilepsy, Alzheimer's disease (AD) and Parkinson's disease are associated with microglial activation (McGeer et al., 1993, 2001; Rothwell and Relton, 1993; Giulian et al., 1996; Sheng et al., 1994). McGeer P L, Yasojima K, McGeer E G (2001). Inflammation in Parkinson's disease. Adv Neurol 86:83-89.

Cytokines (e.g., IL-1, IL-6, and TNF-alpha) and chemokines (e.g., IL-8, MCP-1, MIP-1alpha, MIP-1beta and RANTES) have been implicated in the pathology of numerous conditions and diseases, including secondary cellular damage species, proteolytic enzymes, and a variety of cytokines from MNPs are associated with the initiation and maintenance of tissue damage in the arthritic state (Kunkel et al., J. Leukoc. Biol., 59:6-12, 1996; Badolato and Oppenhiem, Semin. Arthritis Rheum., 2:526-38, 1996).

Protein kinase C (PKC) belongs to a family of serine threonine protein kinases. To date, twelve isoforms in the PKC subfamily have been identified. Kanthasamy et al., 2003; Antioxidants & Redox Signaling, 5:609-620. One such isoform is protein kinase Cdelta (PKCδ). Martelli A M, Mazzotti G, Capitani S. Nuclear protein kinase C isoforms and apoptosis. Eur J Histochem. 2004; 48(1):89-94.

PKCδ was originally discovered by Gschwendt et al. in 1986, Gschwendt M, Kittstein W, and Marks F. A novel type of phorbol ester-dependent protein phosphorylation in the particulate fraction of mouse epidermis. Biochem Biophys Res Commun. 137:766-74, 1986, and cloned from a rat brain cDNA library the following year. Kurkinen K M, Keinanen R A, Karhu R, and Koistinaho J. Genomic structure and chromosomal localization of the rat protein kinase Cdelta-gene. Gene 242:115-23, 2000, Ono Y, Fujii T, Ogita K, Kikkawa U, Igarashi K, and Nishizuka Y. Identification of three additional members of rat protein kinase C family: delta-, epsilon- and zeta-subspecies. FEBS Lett. 226: 125-8, 1987. The PKCδ gene is localized on human chromosome 3, Huppi K, Siwarski D, Goodnight J, and Mischak H. Assignment of the protein kinase C delta polypeptide gene (PRKCD) to human chromosome 3 and mouse chromosome 14. Genomics 19: 161-2, 1994, rat chromosome 16, Kurkinen K M, Keinanen R A, Karhu R, and Koistinaho J. Genomic structure and chromosomal localization of the rat protein kinase Cdelta-gene. Gene 242:115-23, 2000, and mouse chromosome 14, Huppi K, Siwarski D, Goodnight J, and Mischak H. Assignment of the protein kinase C delta polypeptide gene (PRKCD) to human chromosome 3 and mouse chromosome 14. Genomics. 19:161-62 (1994).

Inflammation in the brain is characterized by the activation of microglia and is thought to be associated with the pathogenesis of a number of neurological diseases. Current therapeutic approaches to treating inflammation and disease associated with inflammation in the CNS are lacking. Hence, there is a need for a method to effectively treat inflammatory diseases, disorders or conditions.

### BRIEF SUMMARY OF THE INVENTION

In one aspect, the invention concerns a method for decreasing the amount of one or more pro-inflammatory substances released from an activated microglial cell comprising contacting a microglial cell with an effective amount of a PKCdelta inhibitor.

In another aspect, the invention concerns a method for identifying compounds that inhibit the release of pro-inflammatory substances from microglial cells. The method includes contacting one or more microglial cells with a PKCd inhibitor. The microglial cells may be inactivated microglial cells (quiescent), activated microglial cells, or over-activated microglial cells or combinations thereof. The amount of one or more pro-inflammatory substances released by the microglial cells, preferably activated microglial cells, is determined and compared to a control. A decrease in the amount of pro-inflammatory substances in the presence of the PKCd inhibitor indicates the PKCd inhibitor is a compound that inhibits the release of pro-inflammatory substances.

In still another aspect, the invention concerns a method for identifying compounds that inhibit the production of pro-inflammatory substances within microglial cells. The method includes contacting one or more microglial cells with a PKCd inhibitor. The microglial cells may be unactivated microglial cells, activated microglial cells, or over-activated microglial cells or combinations thereof. The amount of one or more pro-inflammatory substances produced within the microglial cells, preferably activated microglial cells, is determined and compared to a control. A decrease in the amount of pro-inflammatory substances produced in the presence of the PKCd inhibitor indicates the PKCd inhibitor is a compound that inhibits the production of pro-inflammatory substances.

In a further aspect, the invention concerns a pharmaceutical composition for decreasing levels of pro-inflammatory substances in a central nervous system of a mammal in need thereof. The composition includes an effective amount of a protein kinase C delta (PKCd) inhibitor that is effective in decreasing levels of pro-inflammatory substances released from activated microglial cells and a pharmaceutically acceptable carrier.

In yet another aspect, the invention concerns a method for treating or preventing inflammation in the central nervous system of a mammal. The method includes administering to a mammal in need thereof an effective amount of a PKCdelta inhibitor.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE DRAWINGS

The invention can be more fully understood from the following detailed description and the accompanying figures

FIG. 1. Rottlerin attenuates LPS induced release of TNFα. BV-2 microglia were exposed to 1 μg/ml LPS with and without rottlerin (1 to 3 μM) for 16 hrs and cell free supernatants were collected. TNFα levels were measured in Luminex immunoassay. n=6, **p<0.01 compared to controls and ##p<0.01 compared to LPS treated cells.

FIG. 2. siRNA suppression of PKCd attenuates LPS induced release of TNFα. BV-2 microglia were transfected with 20 nM PKCδ siRNA and non-specific siRNA. 24 hr later transfected cells were treated with LPS (1 μg/ml) for 16 hrs and cell free supernatants were collected. Specificity of PKCδ suppression was assessed by western blotting. TNFα levels in the supernatants were determined in Luminex immunoassay. n=6, **p<0.01 compared to controls and ##p<0.01 compared to LPS treated cells.

FIG. 3. LPS induces up regulation and membrane translocation of PKCδ. (A) BV-2 microglia were treated with LPS (1 μg/ml) for 24 hrs and PKCδ expression levels were determined by western blotting. Increased PKCδ expression was observed in LPS treated cells. (B) PKCδ immunofluorescence was performed to visualize membrane translocation in LPS-treated cells using an Alexa-488 conjugated secondary antibody after fixing with 4% paraformaldehyde. Inset. Control panel-Cytosolic and LPS panel-nuclear localization of PKCd are indicated by arrows (60×).

FIG. 4. PKCδ promoter structure and site-directed mutagenesis of NFkB binding sites. (A) The ˜2 kb fragment of mouse PKCδ promoter region (relative to the transcription start site) was cloned using genomic DNA prepared from MN9D cells by the fusion PCR technique. PKCδ promoter fragment was cloned into promoter-less pGL3-basic vector with a luciferase reporter gene (Luc). (B). Site-specific mutagenesis of two NFkB sites present in the PKCδ promoter region.

FIG. 5. Mutation of NFkB sites block TNFa induced PKCδ promoter activity. Briefly, BV2 cells, were transfected with promoter constructs coding for native or NFkB mutated PKCδ promoter (see FIG. 10). 12 h after transfection, cells were exposed to 30 ng/ml TNF with or without etanercept (ETA). 24 h later, cell lsyates were subject to luciferase assay.

FIG. 6. MMP3-induces TNFα release in BV2 cells. BV2 cells were exposed to active and inactive MMP3 (400 ng/ml). After 24 h, the amount of TNFa released into the extracellular medium were determined in Luminex assays (see methods for details). n=6 ** p<0.01 compared to control.

FIG. 7. Immunostaining of primary mouse microglia cells with CD11b+. Primary microglia was isolated from PND2 C57black pups and processed for CD11b immunofluoresence labeling as described in the text.

FIG. 8. Effect of PKCδ inhibitor rottlerin on LPS-induced TNFα release from primary mouse microglia. Primary microglia were treated with 100 ng/ml LPS for 24 hr in the presence of PKCδ inhibitor rottlerin (1 μM and 3 μM). TNFα levels in the culture medium were quantified using a multiplex bead-based luminex® immunoassay. *** p<0.001 compared to controls; ## p<0.05 ### p<0.001 compared to LPS treatment; N=5.

FIG. 9. Intranigral LPS-induced neuroinflammatory animal model. A) Stereotaxic coordinates used in the injection of LPS in the substantia nigra of C57 black mice. B) PKCδ kinase activity measured by the 32P ATP immunoprecipitation kinase assay. **p<0.05 compared to saline; N=3.

FIG. 10. PKCδ inhibitor rottlerin suppresses pro-inflammatory cytokine release in primary mouse microglia. Primary microglia were isolated by differential adherence from post-natal mixed-glial cultures and purity verified to be more than 95% by immunocytochemistry. Primary microglia were treated with LPS (100 ng/ml) for 24 hrs or pre-treated with the PKCδ inhibitor rottlerin (1 μM) and then treated with LPS. After treatment, supernatants were collected and proinflammatory cytokine levels were assayed using a multiplexed luminex assay.

FIG. 11. Purity of primary microglial cultures assessed by double immunofluorescence. Primary microglial cultures obtained by differential adherence were verified to be >95% pure by double immunofluorescence using GFAP as the astrocyte marker and Iba1 as the microglial marker. The nucleus was labeled with Hoechst.

FIG. 12. PKC-delta Lentiviral-shRNA suppresses LPS-induced nitric oxide Release (a marker of proinflammatory effect) in primary mouse microglia. Primary microglia isolated by differential adherence from post-natal mixed-glial cultures were transduced with either PKC-delta or non-specific shRNA expressing lentivirus and treated with LPS for 24 hrs. Supernatants were collected and the nitric oxide release was determined by measuring the amount of released nitrite using the griess assay. PKC δ shRNA and Control shRNA Lentiviral vector were obtained from Santa Cruz Biotechnology, Santa Cruz, Calif. PKC δ shRNA (m) Lentiviral Particles. sc-36246-V (from Santa Cruz Biotechnology 2008-2009 catalog). PKC δ shRNA (m) lentiviral particles are concentrated, transduction-ready viral particles containing a target-specific construct that encodes a 19-25 nt (plus hairpin) shRNA designed to knock down gene expression. Each vial contains 200 μl frozen stock containing 1.0×10⁶ infectious units of virus (IFU) in Dulbecco's Modified Eagle' Medium with 25 mM HEPES pH 7.3. Suitable for 10-20 transductions. Control shRNA lentiviral particles. sc-108080 (from Santa Cruz Biotechnology 2008-2009 catalog). Control shRNA lentiviral particles is a negative control for experiments using targeted shRNA Lentiviral Particle transduction; Control shRNA lentiviral particles encodes a scrambled shRNA sequence that will not lead to the specific degradation of any known cellular mRNA. Each vial contains 200 μl shRNA lentiviral particles sufficient for 10-20 transductions.

FIG. 13. PKC-delta Lentiviral-shRNA suppresses LPS-induced nitric oxide release in mouse midbrain neuron-glia cultures. Primary mouse ventral midbrain neuron glial cultures obtained from E14 mouse brain and were transduced with either PKCdelta shRNA or nonspecific shRNA lentivirus at 4 days in vitro (DW-4) and treated with LPS at DIV10. PKC-delta Lentiviral-shRNA has been described elsewhere herein, for example, in the description of FIG. 12. Supernatants were collected at 48 hrs and nitrite levels were determined using the griess assay.

FIG. 14. Native PKC δ protein is upregulated during microglial activation and is phosphorylated at its activation loop site Thr 505 (pThr 505) in primary microglial cells. Primary microglia isolated by differential adherence from post-natal mixed-glial cultures were treated with LPS (100 ng/ml) or aggregated alpha synuclein (250 nM) that was aged in vitro for 7 days. PKC-delta and phospho PKC-delta Thr-505 levels were determined by western blotting using specific antibodies.

FIG. 15. Activation loop phosphorylation of PKCδ (pThr 505) is highly upregulated in vivo during brain inflammation in the acute MPTP mouse model of Parkinson's Disease. C57BL6 mice were treated were injected with 4 doses of MPTP (18 mg/kg; i.p.) at 2 hr intervals and sacrificed 24 hrs after the last injection. The substantia nigra and striatum were dissected using a brain matrix. Activation loop phosphorylation of PKC-delta was determined using a phospho-specific antibody. Beta actin was used as the loading control and Tyrosine hydroxylase western blotting was used to confirm accurate dissection of the brain regions.

FIG. 16. Primary microglia were isolated from PKC delta knockout (−/−) and wild type (+/+) mice by differential adherence and treated with LPS (0.5 μg/ml) for 24 hr. Proinflamatory cytokines IL-1β, IL-6 and TNFα were determined using a multiplexed luminex immunoassay.

FIG. 17. Primary microglia were isolated from PKC-delta wild type (PKCδ +/+) and knockout (PKCδ−/−) mice and treated with LPS for 24 hrs. Nitric oxide release was determined by measuring the amount of nitrite using the griess assay and a sodium nitrite standard curve. The results indicate that Reduced iNOS (Nitrite Production) in PKCδ −/− microglia.

FIG. 18. Primary microglia from PKCδ knockout mice have attenuated superoxide generation generation (ROS production) FIG. 18. Primary microglia isolated from PKC-delta wild type (PKCδ +/+) and knockout (PKCδ−/−) mice were treated with LPS or TNF alpha for 24 hrs. Intracellular ROS generation was measured using the DCFH-DA dye and the increase in ROS was quantified using the control wells for background subtraction. The results show that primary microglia from PKCδ knockout mice have attenuated superoxide generation generation (ROS production).

FIG. 19. PKC-delta knockdown suppresses key chemokine levels in microglial cells. Primary microglia isolated from PKC-delta wild type (PKCδ +/+) and knockout (PKCδ−/−) mice were treated with LPS for 24 hrs. Supernatants were collected and the chemokines were quantified using the luminex immunoassay system with recombinant standards. The results show that microglia from PKC-delta knockout (−/−) mice have reduced cytokine and chemokine production upon LPS activation.

FIG. 20. PKCdelta wild type (+/+) and knockout (−/−) mice were treated were injected with 1 dose of LPS (5 mg/kg; i.p.) and sacrificed 24 hrs later. The substantia nigra and striatum were dissected using a brain matrix. Cytokine levels in the brain tissue was determined using quantitative SYBR green real time PCR. Western blots for the microglial marker iba1 and iNOS were also used to determine microglial activation. The results show that PKCδ knockout mice have reduced cytokine production and microglial activation in the mouse SNpc and striatum following sSystemic LPS challenge.

FIG. 21. PKCdelta wild type (+/+) and knockout (−/−) mice were treated were injected with 1 dose of LPS (5 mg/kg; i.p.) and behavioral studies were performed 48 hours later using the versamax infrared analyzer. Vertical and horizontal activity amongst other parameters were measured and the data was quantified and expressed graphically. The results show that PKCδ knockout mice are resistant to systemic LPS-induced sickness behavior and motor deficits.

FIG. 22. PKCdelta wild type (+/+) and knockout (−/−) mice were treated were injected with 1 dose of LPS (5 mg/kg; i.p.) and serum was collected 48 hours later by cardiac puncture. Serum cytokines and chemokines were quantified using the luminex immunoassay system. The results show that PKCδ knockout mice have reduced levels of serum cytokines and chemokines following systemic LPS challenge.

FIG. 23. PKCdelta wild type (+/+) and knockout (−/−) mice were treated were injected with 1 dose of LPS (5 mg/kg; i.p.) and serum was collected 48 hours later by cardiac puncture. Serum cytokines and chemokines were quantified using the luminex immunoassay system. The results indicate that PKCδ knockout mice have reduced levels of serum cytokines and chemokines following systemic LPS challenge.

### DETAILED DESCRIPTION OF THE PREFERRED EMBODIMENT

The present invention now will be described more fully hereinafter with reference to the accompanying examples, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the invention set forth herein will come to mind to one skilled in the art to which this invention pertains, having the benefit of the teachings presented in the descriptions and the drawings herein. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

The articles “a” and “an” are used herein to refer to one or more than one (i.e., to at least one) of the grammatical object of the article. By way of example, “an element” means one or more than one element.

The terms protein kinase C delta, PKC-delta, PKCdelta, PKCd, PKCδ or PKC delta are used interchangeably herein.

### Definitions:

As used herein, the term “PKCd inhibitor” includes any compound capable of downregulating, decreasing, reducing, suppressing or inactivating the amount and/or activity of protein kinase C delta (PKCd). Generally, said inhibitors may be proteins, oligo- and polypeptides, polynucleotides, genes, lipid, polysaccharide, drugs, small chemical molecules, or other chemical moieties. Inhibitors for use with the invention may function to inhibit PKCd by any number of ways, including decreasing PKCd mRNA or protein levels or by blocking the activation of PKCd or its activity, for example, through inhibiting or decreasing proteolytic cleavage of PKCd, e.g. using a PKCd peptide cleavage inhibitor, and/or inhibiting the phosphorylation of PKCd, e.g. at tyrosine residues located at positions 221, 570, 813, 1007 and/or 1008 in wild-type PKCd protein, at threonine residue located at position 505 in wild-type PKCd protein, and at serine resides located at positions 643 and 662 in wild-type PKCd protein. Compounds that decrease activity of PKCd downstream of PKCd in its pathway, such as kinases downstream of PKCd, and/or decrease products or activity of PKCd targets, for example, PP2A and TH, or decrease activity upstream of PKCd are also within the scope of PKCd inhibitors of the present invention.

As used herein, the term “neuroinflammation” or “neuroinflammatory diseases, disorders or conditions” refers to diseases, disorders or conditions associated with, caused by or related to inflammation in the central nervous system (CNS).

As used herein, the term “neurodegeneration” refers the damage or death of a cell in the central nervous system, for example, a neuron. Neurodegeneration refers to any pathological changes in neuronal cells, including, without limitation, death or loss of neuronal cells and any changes that precede cell death. The pathological changes may be spontaneous or may be induced by any event and include, for example, pathological changes associated with apoptosis. The neurons may be any neurons, including without limitation sensory, sympathetic, parasympathetic, or enteric, e.g. dorsal root ganglia neurons, motorneurons, and central neurons, e.g. neurons from the spinal cord.

As used herein, the terms “neurodegenerative disorder” or “neurodegenerative disease” refer broadly to disorders or diseases that affect the nervous system having damage or death of a cell of the central nervous system, including but not limited to Parkinson's disease, Alzheimer's disease, Huntington's disease, stroke, ischemia, epilepsy, amyotrophic lateral sclerosis and the like.

As used herein, the term “mammal” includes humans, as well as non-human mammals, cows, pigs, monkeys, horses, dogs, cats, mice, rats, and non-domestic animals. It is contemplated that the methods and compositions herein may be used to treat any suitable animal or mammal, including but not limited to those that are susceptible to neuroinflammation, or are susceptible to any of the other diseases, disorders or conditions described herein, or would benefit from any of these treatments or would serve as appropriate in vivo models.

As used herein, the term “microglial cell” refers to the macrophage like glial cells found in the central nervous system which release pro-inflammatory substances when activated and includes mononuclear phagocytes and macrophages.

As used herein, the term “mononuclear phagocyte” is an immune cell found in blood and body tissues, including the central nervous system and brain, and include, for example, microglia cells, nionocytes, macrophages, histiocytes, dendritic cells, precursor cells of microglia, precursor cells of monocytes, precursor cells of macrophages, microglia-like cell lines, macrophage-like cell lines, or cell lines

As used herein, the term “compound” refers to a polynucleotide, a protein, a polypeptide, a peptide, an antibody, an immunoglobulin, a ligand, a cytokine, a growth factor, a nucleic acid, a lipid, membrane, a carbohydrate, a drug, a prodrug, or a small molecule or a fragment thereof.

As used herein, the term “modulates” refers to an increase or decrease as compared to a control, for example, a PKCd inhibitor may decrease an activity, expression level, symptom, condition, or progression of a disease or disorder associated with PKCd activity or expression as compared to a control or that which would occur in the absence of the PKCd inhibitor, for example, the ability to increase or decrease the amount of pro-inflammatory substances produced within or released from a cell, such as a microglial cell.

As used herein, the term “inhibiting” or “inhibits” refers to the ability to reduce or decrease a level, an amount, an activity, the severity of a disease, disorder, condition or symptom and the like, e.g. of mRNA or protein or activity, for example, as compared to a control.

As used herein, the term “neurotoxin” or “neurotoxicant” includes a substance that injures, damages, or kills a neuron.

As used herein, the term “pharmaceutically acceptable carrier” refers to any carrier, diluent, excipient, wetting agent, buffering agent, suspending agent, lubricating agent, adjuvant, vehicle, delivery system, emulsifier, disintegrant, absorbent, preservative, surfactant, colorant, flavorant, or sweetener, preferably non-toxic, that would be suitable for use in a pharmaceutical composition.

As used herein, the terms “pharmaceutically effective” or “therapeutically effective” shall mean an amount of a PKCd inhibitor that is sufficient to show a meaningful patient benefit, i.e., treatment, prevention, amelioration, or a decrease in the frequency of the condition or symptom being treated. The terms “patient”, “subject” and “recipient” are used interchangeably herein. The PKCd inhibitor may be administered in the form of a pharmaceutical composition with a pharmaceutically acceptable carrier. The term “effective amount” means a dosage sufficient to provide treatment for the disease, disorder or condition being treated. For example, an effective amount of the PKCdelta inhibitor can be an amount that decreases the release of pro-inflammatory substances from activated microglial cells compared to that which would occur in the absence of the PKCdelta inhibitor and may treat, prevent, ameliorate, or decrease a frequency of the condition or symptom associated with or resulting from the release of pro-inflammatory substances such as neuroinflammation.

As used herein, the term “anti-inflammatory substances” refers to substances produced within and/or released from a microglial cell, macrophage, or mononuclear phagocyte, preferably an activated microglial cell, mononuclear phagocyte or macrophage, that decrease inflammation. Anti-inflammatory substances include but are not limited to cytokines such as IL-IRA, TGF-beta, and IL-10. See for example, TGF-beta superfamily members promote survival of midbrain dopaminergic neurons and protect them against MPP+ toxicity. EMBO J. 1995 Feb. 15; 14(4). 736-742; TGFβ3 and TGFβ3 are potent survival factors for midbrain dopaminergic neurons. Neuron, 1 November 1994, Volume 13, Issue 5, 1245-1252

As used herein, the term “pro-inflammatory substances” refers to substances produced within and/or released from a microglial cell, mononuclear phagocyte, or macrophage, preferably an activated microglial cell, mononuclear phagocyte, or macrophage that promote inflammation.

As used herein, the term “treating” refers to. (i) preventing a disease, disorder or condition from occurring in an animal or human that may be predisposed to the disease, disorder and/or condition but has not yet been diagnosed as having it; (ii) inhibiting the disease, disorder or condition, i.e., arresting its development; and/or (iii) relieving the disease, disorder or condition, i.e., causing regression of the disease, disorder and/or condition. For example, as indicated above, treating does not necessarily indicate a reversal or cessation of the disease, disorder or condition afflicting the subject being treated, but could encompass the lessening or reduction in the deleterious signs, symptoms, and/or rate in the progression of the disease, disorder, or condition being treated as compared to that which would occur in the absence of treatment. As understood, a change in the sign, symptom or rate may be assessed at the level of the subject (e.g., the function or condition of the subject is assessed), or at a tissue or cellular level (e.g., the production and/or release of pro-inflammatory substances from activated microglial cells is lessened or reduced). For example, with respect to multiple sclerosis, treatment may be measured by quantitatively or qualitatively to determine the presence/absence of the disease, or its progression or regression using, for example, symptoms associated with the disease or clinical indications associated with the pathology.

As used herein, the term “polypeptide” is interpreted to mean a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. The term “protein” typically refers to large polypeptides. The term “peptide” typically refers to short polypeptides. “Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well-known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADPribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-link formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications. Perspectives and Prospects, pgs. 1 12 in POSTTRANSLATIONAL COVALENT MODIFICATION OF PROTEINS, B. C. Johnson, Ed., Academic Press, New York (1983); Seifter et al., Meth. Enzymol. 182:626 646 (1990) and Rattan et al., Protein Synthesis. Posttranslational Modifications and Aging, Ann. N.Y. Acad. Sci. 663. 48 62 (1992). Polypeptides may be branched or cyclic, with or without branching. Cyclic, branched and branched circular polypeptides may result from post-translational natural processes and may be made by entirely synthetic methods, as well.

As used herein, the term “polynucleotide” is interpreted to mean a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces T.

As provided herein, inhibiting PKCdelta expression or activity can decrease and/or inhibit the release of pro-inflammatory substances from activated microglia cells. Pro-inflammatory substances include but are not limited to cytokines, chemokines, proteases, neurotoxins, prostaglandins, thromboxanes, leukotrienes, or combinations thereof. Exemplary cytokines produced and/or released from activated microglial cells include but are not limited to IL-1, IL-1beta, IL-6, IL-8, IL-10, IL-12, IL-18, IP-10, Tumor Necrosis Factor (TNFα), cyclooxygenase-2, Tumor Necrosis Factor (TNF-beta), and the like. Typical pro-inflammatory cytokines include without limitation IL-1, IL-6, IL-8, IL-12, IL-18, IP-10, Tumor Necrosis Factor (TNFα), cyclooxygenase-2 and the like. Exemplary chemokines produced and/or released from activated microglical cells include but are not limited to IL8, Macrophage Inflammatory Protein (MIP-1α, MIP-1β), Monocyte chemotactic protein (MCP-1), Macrophage antigen complex-1 (MAC1), Stromal cell derived factor-1 (SDF-1), Regulated upon Activation Normal T cell Expressed and Secreted (RANTES) and the like. Exemplary proteases produced and/or released from activated microglical cells include but are not limited to cathepsins B, L, and S, matrix metalloproteinases MMP-1, MMP-2, MMP-3, MMP-9 and the like. Exemplary neurotoxins produced and/or released from activated microglical cells include reactive oxygen species include but are not limited to superoxide, nitric oxide, peroxynite, hydrogen peroxide and the like. Exemplary cytokines produced and/or released from activated microglial or MNP cells include but are not limited to INF-gamma, IL-1, IL-1beta, IL-6, IL-8, IL-10, IL-12, IL-18, IP-10, Tumor Necrosis Factor (TNFα), cyclooxygenase-2, Tumor Necrosis Factor (TNF-beta) and the like. Exemplary prostaglandins produced and/or released from activated microglial cells include but are not limited to Prostaglandin E2 (PGE2), PGD2, PGD2, PHH2 (H2), and the like. It is also contemplated that contacting microglial cells with a PKCd inhibitor will decrease or inhibit prostaglandin-mediated, thromboxane-mediated and/or leukotriene-mediated microglial activation. Exemplary leukotrienes produced and/or released from activated microglial cells include but are not limited to LTB4, LTA4 and the like. In another embodiment, contacting microglial cells with a PKCd inhibitor will decrease or inhibit the synthesis of prostaglandins, thromboxanes and/or leukotrienes from arachidonic acid (AA), e.g. through cycloxygenases (COX) or lipoxygenases (LOX), for example, in microglial cells. In another aspect, contacting microglial cells with a PKCd inhibitor will decrease or inhibit the release of prostaglandins, thromboxanes and/or leukotrienes therefrom.

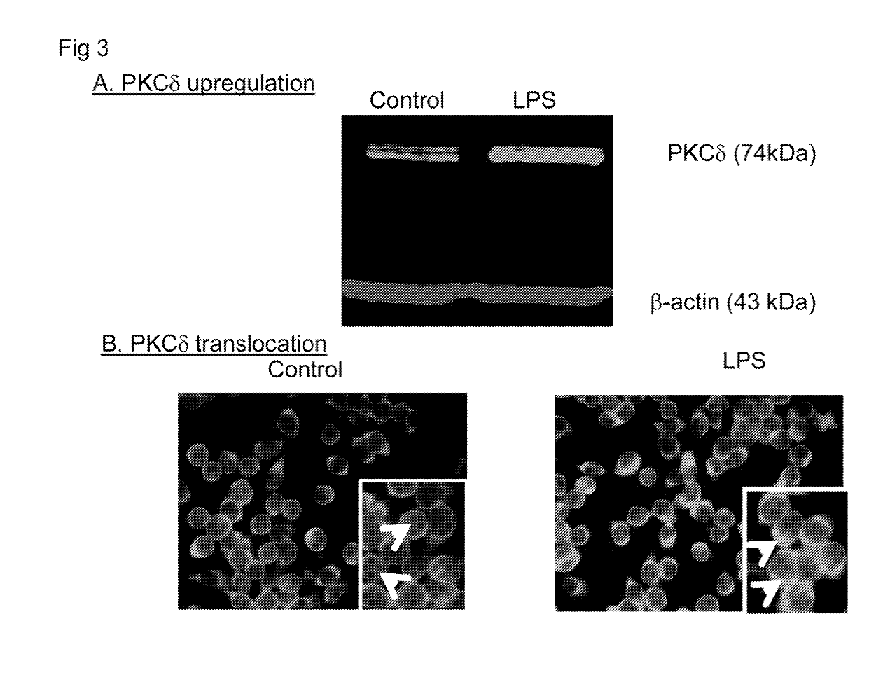

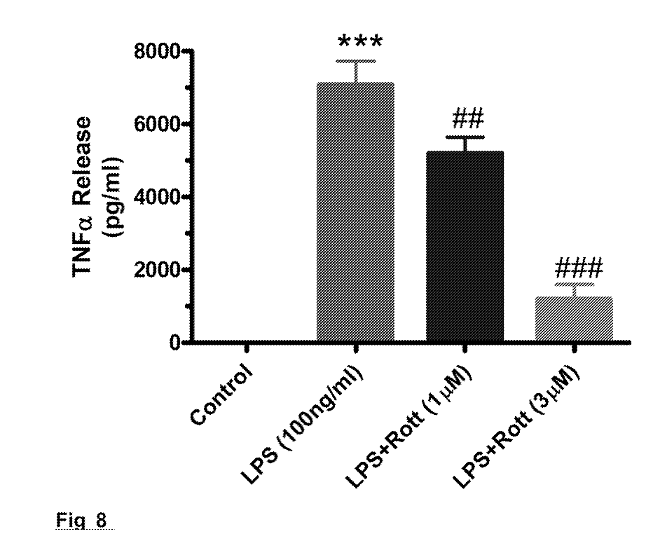

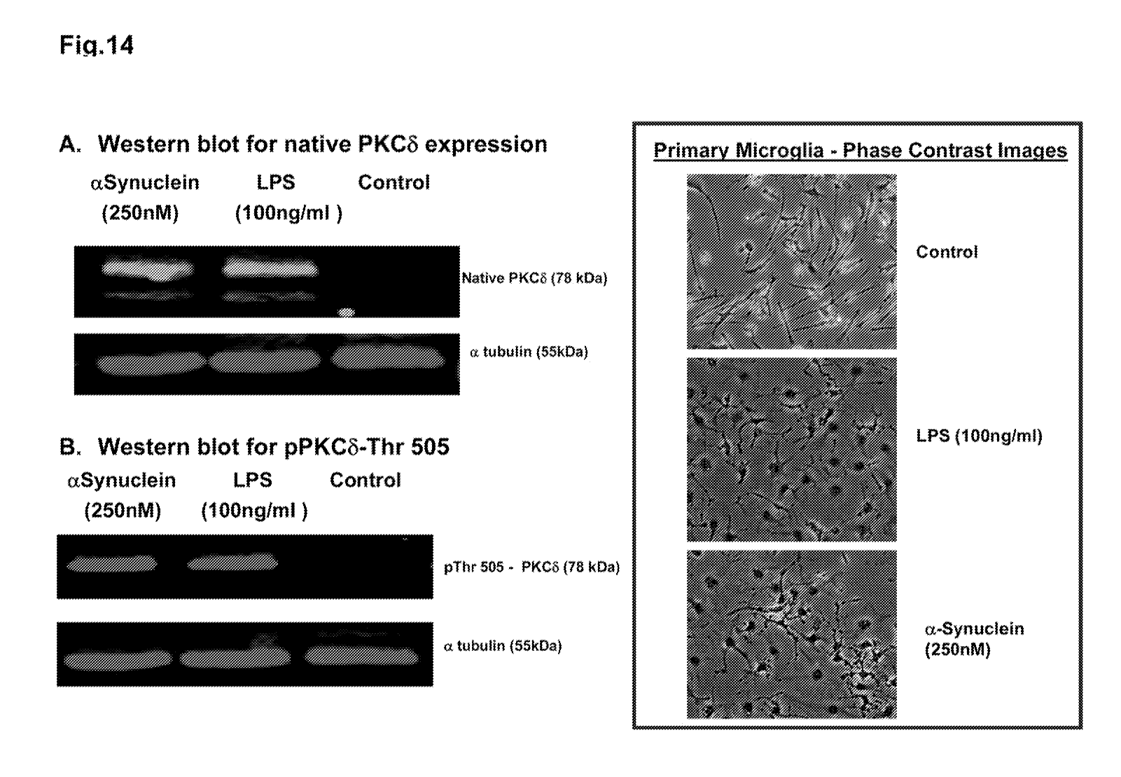

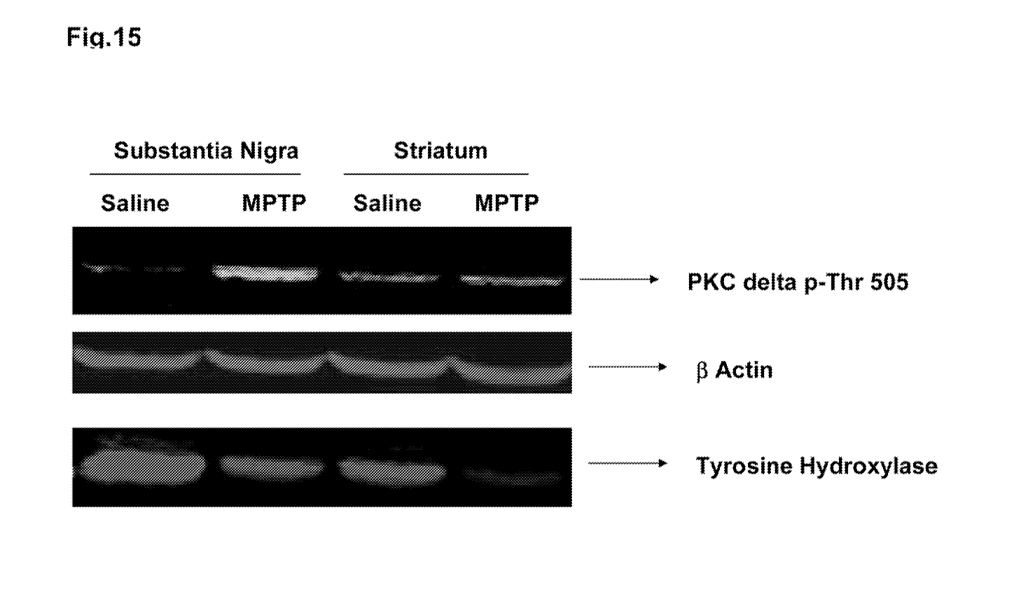

Example 7 herein shows that PKCdelta has upregulated expression and/or activation in activated microglial cells. While not intending to limit the scope of the invention to any particular embodiment, indirect immunofluorescence with an anti-PKCδ polyclonal antibody revealed a dramatic translocation of PKCδ from the cytosol to the plasma membrane in LPS treated BV-2 cells as compared to control cells (FIG. 3B). Unlike dopaminergic neuronal cells, no nuclear translocation was observed in BV-2 cells, demonstrating a distinct mode of PKCδ activation in microglia cells vs neuronal cells in response to neuroinflammatory insult. The phosphorylation of PKCdelta at its activation loop site at amino acid residue threonine position 505 in activated microglial cells indicates the possibility of enhanced PKCdelta activity. See FIGS. 14 and 15. Phosphorylated PKCdelta can be distinguished from unphosphorylated PKCdelta by Western blot analysis as shown in FIG. 14. Immunoblotting for PKCdelta expression in control cells revealed little or no detection of PKCdelta, much less the Thr505 phosphorylated form of PKCdelta. Following LPS stimulation, expression of native and Thr505 PKCdelta was detectable in microglial cells. As described in Example 4, PKCdelta expression may be increased by activation of NFKappa-beta (NFKB) in TNF-alpha activated microglical cells. Importantly, PKCdelta inhibitors show a statistically significant dose-dependent reduction in the release pro-inflammatory substances from LPS activated microglia. For example, the PKCdelta inhibitors, rottlerin and PKCdelta small hairpin RNA, decrease the release of TNF-alpha, IL-12, IL-6 from LPS-stimulated primary mouse microglia and nitric oxide from LPS-stimulated primary mixed glial cultures respectively. See Examples 7-8 and FIG. 8.

The present invention provides a method for inhibiting or treating CNS inflammation associated with activated microglia and the release of pro-inflammatory substances, such as pro-inflammatory cytokines. Accordingly, the present method involves administering to a patient in need thereof an effective amount of a PKCd inhibitor. Although rottlerin or PKCdelta shRNA was used in the Examples herein, other PKCdelta inhibitors may also be used.

Accordingly, the present invention includes a method of inhibiting from microglial cells the release of pro-inflammatory substances, for example, by contacting the microglial cell with a PKCd inhibitor, thereby inhibiting the release of pro-inflammatory substances. The microglial cell may be contacted with the PKCd inhibitor in vitro, ex vivo or in vivo. The method includes contacting one or more microglial cells with one or more PKCd inhibitors. The microglial cells may be unactivated microglial cells, activated microglial cells, or over-activated microglial cells or combinations thereof. The microglial cell may be exacerbated into a state of over-activation upon exposure to further injury or inflammation. Microglia may be activated by any number of substances or actions as known to one skilled in the art and as described elsewhere herein, including for example, LPS, TNF-alpha, LPS, B-amyloid, interferon-gamma, MMP-3, thrombin or inflicted trauma. Use of the methods and compositions described herein can decrease in a mammal the levels of pro-inflammatory substances produced within and/or released from activated microglial cells. Accordingly, methods of the present invention can be used in the treatment of a mammal at risk for or suffering from a disease, disorder or condition associated with the release of pro-inflammatory substances from microglial cells, e.g. activated microglial cells. Without wishing to be bound by this theory, it is contemplated that PKCd inhibitors can reduce the production and/or release of pro-inflammatory substances from microglial cells as well as increase the production and/or release of anti-inflammatory substances, such as anti-inflammatory cytokines. Typical anti-inflammatory cytokines include without limitation IL-10, TGF-beta, and IL-1R. The present invention also provides methods for treating or preventing in a mammal neuroinflammation using PKCd inhibitors. The mammal may be at risk for or suffering from a disease, disorder or condition characterized by neuroinflammation. Methods of the treatment or prevention of diseases are described elsewhere herein.

Any suitable PKCd inhibitor may be used in conjunction with the methods, assays, and compositions of the present invention so long as it modulates one or more activities of PKCd. As used herein, “PKCd activity” refers to an activity exerted by a native or wild type PKCd protein, polypeptide or portion thereof as determined in vivo, ex vivo or in vitro, according to standard techniques. PKCd activity may include any one of the following activities. (1) modulation of the subcellular location of the PKCd protein, e.g. the translocation of the PKCd to from one subcellular location to another, for example, to the plasma membrane from a different subcellular location, such as the nucleus or cytosol, (2) modulation of the phosphorylation of the PKCd protein, (3) modulation of the PKCd protein to phosphorylate a known PKCd substrate, (4) modulation of the expression level of the PKCd mRNA or protein, (5) modulation of the production of pro-inflammatory substances in a microglial cell such as an activated microglial cell, (6) modulation of the release of pro-inflammatory substances from an activated microglial cell, (7) modulation of the production of pro-inflammatory substances in a macrophage or mononuclear phagocyte such as an activated macrophage or mononuclear phagocyte, (8) modulation of the release of pro-inflammatory substances from an activated macrophage or mononuclear phagocyte, (9) modulation of neuroinflammation, (10) modulation of the production of anti-inflammatory substances in a mononuclear phagocyte, macrophage, or microglial cell, (11) modulation of the release of anti-inflammatory substances in a mononuclear phagocyte, macrophage, or microglial cell, (12) modulation of the production of trophic factors from other brain cells such as astrocytes, macrophages or mononuclear phagocytes, 13) modulation of enzymes involved in microglial, mononuclear phagocyte, or macrophage activation, for example, that participate in the production or regulation of pro-inflammatory substances, such as NADPH oxidase and inducible nitric oxide synthase (inos) or isoforms or components of these enzymes, e.g. p67phox, gp91 phox, p47phox and the like, and/or (14) any combination thereof. As used herein, depending on the context in which it is used, modulation refers to an increase or decrease in an activity, amount or level, or a change in the type or kind of activity present as compared to a control. Preferably the PKCd inhibitor decreases or inhibits expression of and/or an activity of a native or wild type PKCd, for example, as compared to a control.

The PKCd inhibitor may be a drug, polynucleotide, peptide, protein, lipid, polysaccharide, or small molecule and the like. In one aspect, the PKCd inhibitor is (3-[(8-Cinnamoyl-5,7-dihydroxy-2,2-dimethyl-2H-1-benzopyran-6-yl)methyl]-2′,4′,6′-trihydroxy-5-methylaceophenone, also referred to as rottlerin. This compound is commercially available from a number of sources including Sigma-Aldrich (St. Louis, Mo.). Also included are rottlerin derivatives and salts thereof, for example, as described in WO2006060196.

In another aspect, the PKCd inhibitor is a polynucleotide that includes but is not limited to an antisense polynucleotide, ribozyme, RNA interference (RNAi) molecule, small hairpin RNA (shRNA), triple helix polynucleotide and the like, where the nucleotide sequence of such polynucleotides are the nucleotide sequences of DNA and/or RNA. Antisense technology may be used to achieve PKCd-specific interference, using for example, stoichiometric amounts of single-stranded nucleic acid complementary to the messenger RNA of PKCd which are introduced into the cell.

In one embodiment, an RNA interference (RNAi) molecule is used as a PKCd inhibitor, decreasing PKCd gene expression in a cell. In another aspect, the PKCd inhibitor is a siRNA molecule for targeting PKCd in a mammal, including without limitation, PKCd siRNA for a mouse, rat, monkey, or human. In one aspect, the PKCd siRNA comprises the sequence of siRNA-d-1. antisense. 5′-AAGATTCACTACATCAAGAACCCTGTCTC-3′, sense. 5′-AAGTTCTTGATGTAGTGAATCCCTGTCTC-3′. In one aspect, the PKCd siRNA comprises the sequence of siRNA-d-2: Antisense. 5′-AAGGTACTTTGCAATCAAGTACCTGTCTC-3′, sense. 5′-AATACTTGATTGCAAAGTACCCCTGTCTC-3′. In one aspect the PKCd siRNA comprises the sequence of siRNA-d-3. Antisense. 5′-AACATCAGGCTTCACCCCTTTCCTGTCTC-3′, sense. 5′-AAAAAGGGGTGAAGCCTGATGCCTGTCTC-3′. In one aspect the PKCd siRNA comprises the sequence of siRNA-d-4. Antisense. 5′-AACTGTTTGTGAATTTGCCTTCCTGTCTC-3′, sense. 5′-AAAAGGCAAATTCACAAACAGCCTGTCTC-3′. In another aspect, an siRNA molecule of siRNA-d-1, siRNA-d-2, siRNA-d-3, and siRNA-d-4 as described above may be used to target rat PKCd. In one aspect, the PKCd siRNA comprises the sequence of siRNA-1. sense strand. 5′-AAUCCACUACAUCAAGAACUU-3′, antisense strand. 5′-GUUCUUGAUGUAGUGGAUUUU-3′. In one aspect, siRNA molecules for the targeting human PKCd comprises the sequence of siRNA-1. sense strand. 5′-AAUCCACUACAUCAAGAACUU-3′, antisense strand. 5′-GUUCUUGAUGUAGUGGAUUUU-3′. The mRNA and amino acid sequence of human PKCd can be found in GenBank accession number. NM_{—}006254 and accession number: NP_{—}997704 respectively. In one aspect, the PKCd siRNA comprises the sequence of siRNA-2. sense. 5′-CUGUGUGUGAAUCUGCUUUUU-3′, antisense. 5′-AAAGCAGAUUCACACACAGUU-3′. In one aspect, the PKCd siRNA for targeting human PKCd comprises the sequence of siRNA-2. sense. 5′-CUGUGUGUGAAUCUGCUUUUU-3′, antisense. 5′-AAAGCAGAUUCACACACAGUU-3′. In one aspect, the PKCd siRNA comprises the sequence of siRNA-1A. Sense strand. 5′-GCAUCUCCUUCAAUUCCUAUUU-3′, antisense strand. 5′-AUAGGAAUUGAAGGAGAUGCUU-3′. In one aspect, the PKCd siRNA comprises the sequence of siRNA-2A. sense strand. 5′-GCAGUUUCUACACAGCAAAGGUU-3′, antisense strand. 5′-CCUUUGCUGUGUAGAAACUGCUU-3′. In one aspect, the PKCd siRNA comprises the sequence of siRNA-3A. sense. 5′-GCCUCACCGAUUCAAGGUUUAUU-3′, antisense. 5′-UAAACCUUGAAUCGGUGAGGCUU-3′. In another aspect, an siRNA molecule of siRNA-1A, siRNA-2A, and siRNA-3A as described above may be used to target mouse PKCd. In one embodiment, the compositions and methods of the present invention includes a PKCd inhibitor of at least one PKCd siRNA. In one aspect, the PKCd inhibitor includes a combination of differing PKCd siRNA molecules. Materials and methods to produce PKCd siRNA molecules are known to one in the art and additionally are described in Kanthasamy, A. G. et al. Suppression of caspase-3-dependent proteolytic activation of protein kinase C delta by small interfering RNA prevents MPP+-induced dopaminergic degeneration. Mol Cell Neurosci. 2004. 25(3):406-21, herein incorporated by reference in its entirety. The PKCd siRNA molecules may be produced by a number of methods, including the use of commercially available kits, for example, The SILENCER™ siRNA Construction Kit (Ambion, Austin, Tex.), a mammalian siRNA PKCd expression plasmid (Upstate Cell Signaling Solutions, Charlottesville, Va.), or obtained from MoleculA (Columbia, Md.).

The siRNA can be administered directly, for example, intracellularly, into a cell to mediate RNA interference (Elbashir et al., 2001, Nature 411:494 498) or administered extracellularly into a cavity, interstitial space, into the circulation of an organism, introduced orally, or may be administered by contacting the cell with a solution containing the RNA. Physical methods of introducing polynucleotides, for example, injection directly into the cell or extracellular injection into the organism, may also be used. Other methods known in the art for introducing polynucleotides to cells may be used, such as viral vectors, viruses, lipid-mediated carrier transport, chemical-mediated transport, such as calcium phosphate, electroporation, and the like. siRNA can be made using, for example, chemical synthesis or in vitro or in vivo transcription. A number of expression vectors have also been developed to continually express siRNAs in transiently and stably transfected mammalian cells (Brummelkamp et al., 2002 Science 296:550-553; Sui et al., 2002, PNAS 99(6):5515-5520; Paul et al., 2002, Nature Biotechnol. 20:505-508

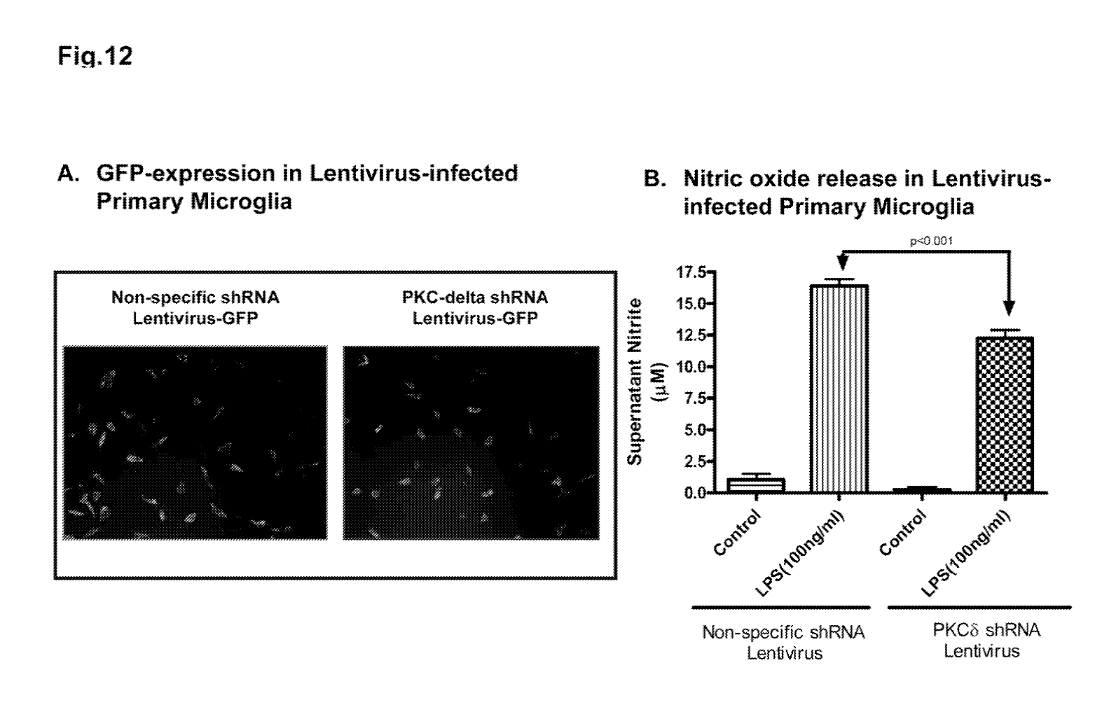

In one embodiment, the PKCd inhibitor is a small hairpin RNA (shRNA), which is processed in vivo into siRNA-like molecules capable of carrying out gene-specific silencing. See, for example, FIG. 12. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition; lower doses may also be useful for specific applications. The RNA molecule may be at least 10, 12, 15, 20, 21, 22, 23, 24, 25, or 30 nucleotides in length.

RNA containing a polynucleotide sequence identical to a portion of PKCd gene is preferred for inhibition. RNA sequences with insertions, deletions, and single point mutations relative to the target sequence of PKCd may also be effective for inhibition. Thus, one hundred percent sequence identity between the RNA and the target gene is not required to practice the present invention. Greater than 80% or 90% sequence identity or 100% sequence identity, between the inhibitory RNA and the portion of the PKCd is preferred. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Alternatively, the duplex region of the RNA may be defined functionally as a nucleotide sequence that is capable of hybridizing with a portion of the PKCd transcript (e.g., 400 mM NaCl, 40 mM PIPES pH 6.4, 1 mM EDTA, 50° C. or 70° C. hybridization for 12-16 hours; followed by washing). The length of the identical nucleotide sequences may be at least 25, 50, 100, 200, 300 or 400 bases.

In one aspect, the PKCd inhibitor is a PKCd peptide cleavage inhibitor. In one aspect of the present invention, a PKCd inhibitor is a PKCd peptide cleavage inhibitor in which the inhibitor includes the following amino acid motif Asp Ile Pro Asp, (Aspartic Acid, Isoleucine, Proline, and Aspartic Acid). In another aspect, the PKCd inhibitor includes a polynucleotide that encodes a peptide or polypeptide containing the amino acid motif Asp Ile Pro Asp where the translated polypeptide inhibits a PKCd activity as disclosed in U.S. patent application Ser. No. 11/262,677 which is herein incorporated by reference in its entirety.

In another embodiment of the present invention, the PKCd peptide cleavage inhibitor is chemically modified to protect the inhibitor from protease degradation. Methods to prevent the protease degradation are known to one skilled in the art, including the addition of N-benzyloxycarbonyl at the N-terminal of the polypeptide. In yet another embodiment, the PKCd peptide cleavage inhibitor is chemically modified to contain a (O-methyl)fluoromethyl ketone (FMK) tail. Tails may facilitate the inhibitor in permeating cell membranes. Other tails that facilitate cell membrane permeability are well known in the art. These include, but are not limited to, N-Acetyl or chloromethyl ketone derivatives. In one embodiment, the PKCd inhibitor is N-benzyloxycarbonyl-Asp(OMe)-Ile-Pro-Asp(OMe)-FMK. Peptides can be synthesized by methods known to one skilled in the art or isolated from cells or tissues of organisms using standard methods known in the art.

The incomplete cleavage of PKCδ can be measured using standard techniques known to one skilled in the art. In another embodiment, the PKCd inhibitor prevents the phosphorylation of PKCd, for example, at tyrosine residues located at positions 221, 570, 813, 1007 and/or 1008 in wild-type PKCd protein, at threonine residue located at position 505 in wild-type PKCd protein, and/or at serine resides located at positions 643 and 662 in wild-type PKCd protein or combinations thereof. In some cases, the PKCd inhibitor inhibits the translocation of PKCd to the plasma membrane from another subcellular location.

In a preferred embodiment, this invention provides methods and assays for identifying compounds that are capable of modulating one or more PKCd activities. The compounds according to this invention preferably inhibit one or more PKCd activities. The methods and assays can be performed in vitro using any suitable cells, such as glial cells, microglial cells, MNP's, macrophages, non-transformed cells, immortalized cell lines, or recombinant cell lines.

In one embodiment, the methods comprise identifying compounds that modulate an activity of PKCd. The method may include contacting a microglial cell with a candidate PKCd inhibitor and determining an alteration in PKCd activity. PKCd activity is described elsewhere herein. The methods include both in vitro, ex vivo, and in vivo screening methods for the ability of the PKCd inhibitor to modulate a PKCd activity, for example, an alteration in post-translational modification (phosphorylation), subcellular location, kinase activity, amount of PKCd, production and/or release of pro-inflammatory substances, production and/or release of anti-inflammatory substances, effects on activated microglial cells, and/or neuroinflammation.

In a preferred embodiment, the method includes identifying a PKCd inhibitor compound that decreases one or more activities of the PKCd. In another embodiment, the method includes identifying a PKCd activator compound that increases one or more activities of the PKCd. Candidate PKCd inhibitors may be evaluated in vitro, in vivo or ex vivo.

In some embodiments, the methods include exposing a microglial cell to an agent to induce activation of the cell. Suitable agents are known in the art and include but are not limited to TNF-alpha, LPS, B-amyloid, interferon-gamma, MMP-3, or thrombin and the like. Activation of the microglial cell, for example, in vivo, may also include activation by infliction of injury, infliction of trauma, viral infection, bacterial infection, exposure to a chemical, exposure to a cytokine, exposure to irradiation, or exposure to conditions that normally result in inflammation. In preferred embodiments, the cells are in an animal model for neuroinflammation. Alternatively, the cells in a cell culture are obtained from an animal model and the effect of the candidate PKCd inhibitor on PKCd activity may be evaluated ex vivo. In some cases, the response to the neuroinflammation in vivo may be determined by edema, higher levels of inflammatory cytokines and microglial, macrophage, or MNP infiltration or localization to the insulted CNS tissue. Detection of the pro-inflammatory substances can be determined in microglial cells or in brain tissue, for example, striatal tissues, using high-performance liquid chromatography. The effect of the methods and PKCd inhibitors, known or candidate PKCd inhibitors, may be assessed at the cellular or tissue level (e.g., histologically or morphometrically), or by assessing a subject's neurological status. Any suitable technique or method for evaluating neuroinflammation or the progression of neuroinflammation may be used including the Griess assay, measurement of proinflammatory cytokine levels (proteins and mRNA levels), immunohistochemical markers of microglial activation (CD11b, iba-1), superoxide generation, nitrotyrosine, flow cytometry, and the like. See, for example, Green L C, Wagner D A, Glogowski J, Skipper P L, Wishnok J S and Tannenbaum S R (1982) Analysis of nitrate, nitrite, and [¹⁵N]nitrate in biological fluids. Anal Biochem 126. 131-138; Liu Y X, Qin L, Wilson B C, An L, Hong J S and Liu B (2002b) Inhibition by naloxone stereoisomers of β-amyloid peptide (1-42)-induced superoxide production in microglia and degeneration of cortical and mesencephalic neurons. J Pharmacol Exp Ther 302. 1212-1219; Liu B, Du L and Hong J S (2000a) Naloxone protects rat dopaminergic neurons against inflammatory damage through inhibition of microglia activation and superoxide generation. J Phar Exp Ther 293. 607-617; Kong L-Y, McMillian M K, Hudson P M, Jin L and Hong J-S (1997) Inhibition of lipopolysaccharide-induced nitric oxide and cytokine production by ultralow concentrations of dynorphins in mixed glia cultures. J Pharmacol Exp Ther 280. 61-66. The reduction of pro-inflammatory substances can be assessed by various methods as would be apparent to those in the art; one such method is to measure the production or presence of compounds that are known to be produced by activated glia, and compare such measurements to levels of the same compounds in control situations. Alternatively, the effects of the present methods and compounds in inhibiting, suppressing, reducing or preventing the release of pro-inflammatory substances from activated microglial cells or on neuroinflammation may be assessed by comparing the signs and/or symptoms of CNS disease in treated and control subjects, where such signs and/or symptoms are associated with or secondary to the production and release of pro-inflammatory substances from activated microglia.

A method of screening a test compound for the ability to decrease the production within or release of pro-inflammatory substances from an activated microglial cell includes contacting a microglial cell with a PKCd inhibitor compound and an agent that is known to activate microglia, such as LPS. As appreciated by one skilled in the art, the agent used to activate microglia may be administered so that the agent contacts the cell prior to the PKCd inhibitor, concurrent with the PKCd inhibitor, or subsequent to the PKCd inhibitor. At least one pro-inflammatory substance is then measured. A decrease in the pro-inflammatory substances (compared to that which occurs in the absence of the PKCd inhibitor compound) indicates that the test compound decreases the release of pro-inflammatory substances. Exemplary pro-inflammatory substances such as cytokines, chemokines, proteases or neurotoxins and their detection are described elsewhere herein. Thus, as will be appreciated by those in the art, there are a number of different assays which may be performed including Griess assays. As mentioned, in vivo the response to the neuroinflammation may be determined by edema, increased levels of pro-inflammatory substances, such as pro-inflammatory cytokines, and microglial or MNP infiltration or localization to the insulted CNS tissue. Detection of the pro-inflammatory substances can be determined in microglial cells or in brain tissue, for example, brain tissues, using luminex ELISA method, qRT-PCR, Western blot, immunohistochemistry, flow cytometry and high-performance liquid chromatography and the like.

See, for example, Hulse et al., 2004 R. E. Hulse, P. E. Kunkler, J. P. Fedynyshyn and R. P. Kraig, Optimization of multiplexed bead-based cytokine immunoassays for rat serum and brain tissue, J Neurosci Methods 136 (2004); Martins et al., 2002 T. B. Martins, B. M. Pasi, J. W. Pickering, T. D. Jaskowski, C. M. Litwin and H. R. Hill, Determination of cytokine responses using a multiplexed fluorescent microsphere immunoassay, Am J Clin Pathol 118 (2002); Khan et al., 2004 S. S. Khan, M. S. Smith, D. Reda, A. F. Suffredini and J. P. McCoy Jr., Multiplex bead array assays for detection of soluble cytokines. comparisons of sensitivity and quantitative values among kits from multiple manufacturers, Cytom B Clin Cytom 61 (2004), pp. 35-39. Thus, in another aspect, the methods of the present invention include identifying PKCd inhibitors that decrease expression of PKCd in an activated microglial cell comprising contacting a microglial cell with a compound (candidate PKCd inhibitor compound) and an agent that activates microglia, measuring the level of PKCd in the microglial cell, and comparing the level of PKCd that occurs in the microglial cell in the presence of the test compound with the level of PKCd that occurs in a microglial cell in the absence of the test compound.

A decrease or reduction in the amount of PKCd, for example, the mRNA or protein level of PKCd, in the presence of the test compound, such as a PKCd siRNA, PKCd shRNA, or antisense PKCd RNA, as compared to the mRNA or protein level of PKCd in the absence of the test compound indicates that the test compound decreases PKCd expression. Modulation of PKCd expression levels can be assayed in a variety of ways known in the art. For example, mRNA levels can be quantitated by, e.g., Northern blot analysis, competitive polymerase chain reaction (PCR), or real-time PCR (RT-PCR). RNA analysis can be performed on total cellular RNA or poly(A)+mRNA. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions.

Protein levels of can be quantitated in a variety of ways well known in the art, such as immunoprecipitation, Western blot analysis (immunoblotting), ELISA or fluorescence-activated cell sorting (FACS). Antibodies directed to PKCd can be identified and obtained from a variety of sources, such as from Santa Cruz Biotechnology, Inc. (Santa Cruz, Calif.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997. Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991. In comparing the levels of PKCd in the presence and absence of the test compound, the compound may decrease in the expression of PKCd mRNA or protein by at least 10%, 20%, 30%, 40%, 50%, 60%, 70%, 80%, 90% or more.

Thus, in another aspect, the method also includes identifying a compound that decreases PKCd activity in a microglial cell comprising. Contacting a microglial cell with a test compound and an agent that activates microglia, determining an effect of the test compound on the kinase activity of the PKCd polypeptide, thereby identifying a compound that modulates the kinase activity of the PKCd polypeptide. In another aspect, the present invention provides for determining the effect of the test compound on the PKCd kinase activity, e.g. determining whether phosphorylation of the PKCd polypeptide and/or activation of PKCd polypeptide in the presence of said compound is changed compared to the phosphorylation and/or activation of the PKCd polypeptide in the absence of said compound. Phosphorylation of PKCd takes place at tyrosine residues located at positions 221, 570, 813, 1007 and/or 1008 in wild-type PKCd protein, at threonine residue located at position 505 in wild-type PKCd protein, and at serine resides located at positions 643 and 662 in wild-type PKCd protein. The inhibition or reduction of PKCd activity can be determined using a variety of methods and assays routine to one skilled in the art, for example, determining the phosphorylation and/or activation of a PKCd kinase's target. Generally, a purified or partially purified PKCd kinase is incubated with a peptide comprising the target sequence of PKCd under conditions suitable for the kinase to phosphorylate its target sequence of amino acids (i.e., protein, polypeptide). The particular requirements of the kinase may be determined empirically by one of skill in the art, or the conditions that have been published for a particular kinase may be used. The extent of phosphorylation of the target peptide is determined in the presence and absence of the test compound and may be determined in the presence of varying concentrations of the test compound. The phosphorylation rate may be determined by any means known in the art including electrophorectic assays, chromatographic assays, phosphocellulose assays and the like.

In an electrophorectic assay, a radiolabled phosphate donor such as ATP or GTP is incubated with the peptide substrate in the presence of a kinase. The phosphorylated substrate versus the phosphate donor (e.g., ATP, GTP) is separated via thin-layer electrophoresis (Hunter J. Biol. Chem. 257:4843, 1982; incorporated herein by reference). Any matrix may be used in the electrophoresis step including polyacrylamide, cellulose, etc. The extent of phosphorylation may then be determined by autoradiography or scintillation counting.

The labeled phosphate donor may be separated from the phosphorylated amino acid sequence by standard chromatography techniques. Any matrix may be used to effect the separation including ion exchange resins, PEI cellulose, silica gel, etc. Standard column chromatography methods may be used, or HPLC methods may be used for faster cleaner separations. The radio-labeled peptides are detected by scintillation counting to determine the phosphorylation rate.

Another method which is historically the most popular is the phosphocellulose paper assay, first described by Witt et al. (Witt et al. Anal. Biochem. 66:253, 1975; incorporated herein by reference). Immunological methods may also be used to detect the phosphorylation of a peptide or protein substrate. For example, anti-phosphotyrosine or anti-phosphoserine antibodies may be used in the detection or precipitation of phosphorylated amino acid sequences. For example, multiple PKCd antibodies that detect the phosphorylated and unphosphorylated forms of PKCd are commercially available. (Cell signaling, Beverly, Mass. and Santa Cruz, Santa Cruz, Calif.).

In comparing the rates of phosphorylation in the presence and absence of the test compound, the compound should lead to at least a 10% decrease in the rate of phosphorylation, more preferably at least 25%, or at least 40%. These decreases are preferably obtained at micromolar concentrations of the compound and more preferably nanomolar concentrations (e.g., less than 100 nM).

In another aspect, the method also includes identifying a compound that modulates the subcellular location of PKCd in a microglial cell comprising. contacting a microglial cell with a test compound and an agent that activates microglia, determining an effect of the test compound on the subcellular location of the PKCd polypeptide, thereby identifying a compound that modulates the subcellular location of the PKCd polypeptide. A reduction or inhibition of the translocation of PKCd to the plasma membrane in the presence of the test compound, as compared to subcellular location of the PKCd the absence of the test compound, for example, from the cytosol, indicates that the test compound modulates PKCd activity. A reduction or inhibition of the translocation of PKCd to the plasma membrane in the presence of the test compound, as compared to subcellular location of the PKCd the absence of the test compound indicates that the test compound may be useful in decreasing the production of pro-inflammatory substances within activated microglial cells, the release of pro-inflammatory substances from activated microglial cells, or in the treatment of neuroinflammatory diseases, disorders or conditions in vitro, ex vivo, or in vivo. The location of the PKCd protein can be determined using a variety of methods and assays routine to one skilled in the art, for example, immunohistochemistry and Western blotting of subcellular fractions isolated by differential centrifugation.

In another aspect, the invention includes determining whether a potential PKCd inhibitor inhibits PKCd kinase activity. In one aspect of the methods of the invention, a PKCd inhibitor decreases PP2A activity. The PKCd inhibitors may decrease Protein Phosphatase 2 (PP2A) activity by inhibiting an activator (phosphorylator) of PP2A, PKCd. In one embodiment, the activity of the PP2A is decreased by at least 10%. In more preferred embodiments, the activity of the phosphatase is decrease by at least 25%, or even more preferably, by at least 50%. Decreased activity of PP2A can be assessed by methods known to one of ordinary skill in the art. Suitable assays are described, for example by Honkanen et al. (1994) Toxicon 32:339 and Honkanen et al. (1990) J. Biol. Chem. 265. 19401. Briefly, phosphatase activity is determined by quantifying the [³²P] released from a ³²P -labeled substrate such as phosphohistone or phosphorylase α. Decreased [³²P] release in the presence of the PKCd inhibitors of the present invention relative to control samples provides a measure of the ability of the PKCd inhibitors of the invention to decrease PP2A activity. In addition to ³²P phosphorylation assays, PP2A phosphatase assays that measure PP2A's phosphatase activity may be used, for example, the serine/threonine phosphatase assay kit from Promega.

In the present invention, methods of identifying and selecting potential therapeutic compounds for their ability to act as a PKCd peptide cleavage inhibitor are contemplated. In one embodiment, the potential PKCd peptide cleavage inhibitor is administered to living cells. If the cell does not contain a protein or polypeptide with a PCKd cleavage site upon which caspase-3 acts enzymatically or caspase-3, then vectors expressing these proteins can be transfected using standard techniques known to one skilled in the art. Therefore, the PKCδ substrate and/or caspase-3 may be endogenous or exogenous in origin. Kaul S, Kanthasamy A, Kitazawa M, Anantharam V, Kanthasamy A G, Caspase-3 dependent proteolytic activation of protein kinase C delta mediates and regulates 1-methyl-4-phenylpyridinium (MPP⁺)-induced apoptotic cell death in dopaminergic cells. relevance to oxidative stress in dopaminergic degeneration. Eur J Neurosci. 2003 September; 18(6):1387-401. (noting that PKCδ can be delivered intracellularly using a lipid-mediated protein delivery system.).

The cleavage of PKCd into two subunits of 41 kDa and a 38 kDa by caspase-3 may be determined using Western blot analysis or enzymatic assays. For example, PKCd enzymatic activity can be assayed using an immunoprecipitation assay as previously described. Reyland M E, Anderson S M, Matassa A A, Barzen K A, Quissell D O. Protein kinase C delta is essential for etoposide-induced apoptosis in salivary gland acinar cells. J Biol Chem. 1999 Jul. 2; 274(27):19115-23., Anantharam V, Kitazawa M, Wagner J, Kaul S, Kanthasamy A G. Caspase-3-dependent proteolytic cleavage of protein kinase Cdelta is essential for oxidative stress-mediated dopaminergic cell death after exposure to methylcyclopentadienyl manganese tricarbonyl. J Neurosci. 2002 Mar. 1; 22(5):1738-51. Kaul S, Kanthasamy A, Kitazawa M, Anantharam V, Kanthasamy A G, Caspase-3 dependent proteolytic activation of protein kinase C delta mediates and regulates 1-methyl-4-phenylpyridinium (MPP⁺)-induced apoptotic cell death in dopaminergic cells. relevance to oxidative stress in dopaminergic degeneration. Eur J Neurosci. 2003 September; 18(6):1387-401) (describing antibodies specific for PKCd).

In another embodiment of the invention, in addition to PKCd peptide cleavage inhibitors, peptides may be prepared and screened to identify those that alter PKCd activity, particularly the ability of PKCd to phosphorylate PP2A or modulate the production within microglial cells and/or release therefrom of pro-inflammatory and/or anti-inflammatory substances, preferably in activated microglial cells. PKCd inhibitors for use with the methods and pharmaceutical compositions of the present invention also include dominant negative mutants, such as a nonphosphorylatable form of PKCd, which directly inhibits PKCd activity by preventing PKCd from phosphorylating normal PKCd in a cell. Dominant negative mutants include loss of function mutants, for example, PKCδ_{D327A} (caspase-cleavage resistant), PKCδ_{K376R} (kinase inactive) and PKCδ_{Y311F} (phosphorylation defective) proteins. See Kaul et al., 2003; Kitazawa et al., 2003; Anantharam et al., 2004; Kaul et al., 2005b; Latchoumycandane et al., 2005.

In one aspect, the PKCd inhibitor is a dominant negative PKCd protein. In one aspect, the dominant negative PKCd is a mouse caspase-cleavage resistant PKCδ^{D327A}-GFP, where an aspartate amino acid residue at position 327 in wild type mouse PKCd (GenBank Accession No. NM_{—}011103) is mutated to an alanine amino acid residue (DeVries T A, Neville M C, Reyland M E, (1999) Nuclear import of PKCdelta is required for apoptosis. identification of a novel nuclear import sequence. EMBO J. 2002 Nov. 15; 21(22):6050-60). The reference is herein incorporated in its entirety.

In one aspect, the PKCd inhibitor is a kinase inactive PKCd. In one aspect, the PKCd is the kinase inactive PKCδ^{K376R}-GFP, where a lysine amino acid residue at position 376 is mutated to arginine amino acid residue compared to wild type mouse PKCd (GenBank Accession No. NM_{—}011103) as described in Li, L., Lorenzo, P. S., Bogi, K., Blumberg, P. M. and Yuspa, S. H. (1999) Protein kinase Cδ targets mitochondria, alters mitochondrial membrane potential and induces apoptosis in normal and neoplastic keratinocytes when overexpressed by an adenoviral vector. Mol. Cell. Biol., 19, 8547-8558, herein incorporated in its entirety.

In one aspect, the dominant negative PKCd is Rat tyrosine 311 phosphorylation defective FLAG-tagged PKCδ^{Y311F} (PKCδ^{Y311F} mutant) where a tyrosine at amino acid residue at position 311 is mutated to phenylalanine amino acid residue in rat PKCδ. (GenBank accession number NM_{—}133307, NP_{—}579841) (Konishi H, Yamauchi E, Taniguchi H, Yamamoto T, Matsuzaki H, Takemura Y, Ohmae K, Kikkawa U, Nishizuka Y. (2001) Phosphorylation sites of protein kinase C delta in H202-treated cells and its activation by tyrosine kinase in vitro. Proc Natl Acad Sci USA. 2001 Jun. 5; 98(12):6587-92), herein incorporated in its entirety.

A nucleic acid molecule encoding a PKCd dominant negative mutant, as discussed above, can be expressed from a vector, which is introduced into a cell in which it is desired to express the dominant negative mutant. An expression vector expressing, for example, a PKCd inhibitor polynucleotide can be introduced into cells using well known transfection methods (see, for example, Sambrook et al., Molecular Cloning. A laboratory manual (Cold Spring Harbor Laboratory Press 1989); Ausubel et al., Current Protocols in Molecular Biology (Green Publ., N.Y. 1989), each of which is incorporated herein by reference).

A vector also can contain elements required for replication in a prokaryotic or eukaryotic host system or both, as desired. Such vectors, which include plasmid vectors and viral vectors such as bacteriophage, baculovirus, retrovirus, lentivirus, poliovirus, rhinovirus, vaccinia virus, influenza virus, adenovirus, adeno-associated virus, herpes simplex virus, measles coronavirus, Sindbis virus, and semliki forest virus vectors, are well known and can be purchased from a commercial source (Promega, Madison, Wis.; Stratagene, La Jolla, Calif.; GIBCO/BRL, Gaithersburg, Md.) or can be constructed by one skilled in the art (see, for example, Meth. Enzymol., Vol. 185, D. V. Goeddel, ed. (Academic Press, Inc., 1990); Jolly, Canc. Gene Ther. 1:51-64 (1994); Flotte, J. Bioenerg. Biomemb. 25:37-42 (1993); Kirshenbaum et al., J. Clin. Invest 92:381-387 (1993), which is incorporated herein by reference).

In another aspect, the PKCd inhibitor may be a drug, for example, using drugs from a compound library. Thus, PKCd inhibitors include those compounds known to inhibit PKCd activity, for example, (3-[(8-Cinnamoyl-5,7-dihydroxy-2,2-dimethyl-2H-1-benzopyran-6-yl)methyl]-2′,4′,6′-trihydroxy-5-methylaceophenone (rottlerin), dominant negative mutants, or those known to decrease expression of PKCd, for example, PKCd siRNA, or those compounds yet to be identified, for example, those identified using the screening methods described herein. The efficacy of a PKCd inhibitor for decreasing PKCd activity or PKCd expression levels may be assayed in vitro or in vivo according to methods known in the art and as described herein. See, for example, PKCdelta inhibitor rottlerin protects nigral dopaminergic neuronal death in MPTP-induced animal model of Parkinson's disease (Zhang D, Anantharam V, Kanthasamy A, Kanthasamy A G., Neuroprotective effect of protein kinase C delta inhibitor rottlerin in cell culture and animal models of Parkinson's disease, J Pharmacol. Exp Ther. 2007 September; 322(3):913-22).

The methods and assays of this invention are designed to detect the presence of increased or decreased levels of pro-inflammatory substances produced with a microglial cell, increased or decreased levels of pro-inflammatory substances released from an activated microglial cell, increased or decreased levels of neuroinflammation upon treatment with a candidate PKCd inhibitor compound or a known PKCd inhibitor.

According to one embodiment, the assay is performed by contacting glial cells, e.g. microglial cells, with a known or candidate PKCd inhibitor and a suitable activation agent. Any suitable cells may be used in the methods and assays described herein. According to one aspect, the cells are mouse, rodent, or human glial cells. Preferably, the cells are activated microglial cells. The amount of one or more pro-inflammatory substances released by the glial cells is measured, and then compared to the amount of pro-inflammatory substances released in the absence of the test compound or in the presence of a different amount of the test compound. The pro-inflammatory substances may include one or more of the following. IL-1, IL-6, IL-8, IL-10, IL-12, IL-18, IP-10, Tumor Necrosis Factor (TNFα), cyclooxygenase-2, Tumor Necrosis Factor (TNF-beta), IL8, Macrophage Inflammatory Protein (MIP-1α, MIP-1β), Monocyte chemotactic protein (MCP-1), Macrophage antigen complex-1 (MAC1), Stromal cell derived factor-1 (SDF-1), Regulated upon Activation Normal T cell Expressed and Secreted (RANTES), cathespins B, L, and S, the matrix metalloproteinase MMP-1, MMP-2, MMP-3, MMP-9, a reactive oxygen species such as a superoxide, nitric oxide, peroxynite, or hydrogen peroxide or other cytokine, chemokine, protease, neurotoxin, prostaglandin, thromboxane, leukotriene or any combinations of these. The amount or level of pro-inflammatory substances within the activated microglial cell or released from the microglial cell may be determined by any suitable method, including ELISA, Griess assay, flow cytometry, for example, using flow cytometry to isolate Brain microglia by F4/80⁺, CD11b⁺), luminex assay and other microbead assays.

A further assay according to another embodiment includes an animal model of an inflammatory disease, to which a compound that modulates PKCd activity, preferably a PKCd inhibitor, is administered. The effects of the neuroinflammation in the animal model are measured. The neuroinflammatory disease may be Parkinson's disease, Lewy body dementia, traumatic brain injury, tauopathies, prion disease, vascular dementia, or a combination of these. The effects can be assessed qualitatively or quantitatively, or both. The presence or absence of neuroinflammation or neuroinflammatory activity may be determined using immune cell infiltration, such as the presence of polymorphonuclear neutrophils or macrophages and are known to one skilled in the art and are contemplated by the present invention. Neuroinflammation can also be detected using various standard neuroinflammatory techniques. See, for example, Protocol for Neural Cell Culture (3rd Edition), Ed. S. Fedoroff, A. Richardson. (2001); Neuroinflammation—From Bench to Bedside. Helmut Kettenmann, G. A. Burton, U. J. Moenning. Volume 39 of Ernst Schering Research Foundation workshop (Springer 2002); Neuroglia in the aging brain. Jean De Vellis. (Humana Press, 2002); Neuroinflammation. Mechanisms and management. Paul L. Wood. (Humana Press, 2003).

Methods and pharmaceutical compositions of the present invention may be used to modulate, i.e. increase or decrease, the levels of proinflammatory substances or neuroinflammation or both in the central nervous system of a mammal. The invention includes a method of decreasing levels of proinflammatory substances or neuroinflammation or both in the central nervous system in a mammal in need thereof by administering an effective amount or therapeutically effective amount of a PKCd inhibitor. Preferably, the compound or known PKCd inhibitor decreases the mRNA or protein level of the one or more pro-inflammatory substances produced within the microglial cell or released by the microglial cell by at least 10%, 20%, 30%, 40%, 50%, 60%, 70%, 80%, 90% or more as compared to a control. Preferably the microglial cell is an activated microglial cell.

Use of the methods and compounds according to the present invention are indicated for treating, ameliorating, or preventing certain signs, symptoms, and/or deleterious neurological effects associated with the release of pro-inflammatory substances from activated microglia. A patient may be assessed over time for the appearance, absence, or progression of disease symptoms (e.g., weekly, monthly, annually, or semi-annually) or pro-inflammatory substances. In another embodiment, the method includes inhibiting in a mammal the release of pro-inflammatory substances from activated microglial cells.

Accordingly, provided herein are methods for the treatment of diseases, disorders or conditions associated with the release of pro-inflammatory substances from microglial cells, e.g. activated microglial cells.

Inflammation and pro-inflammatory substances contribute to acute or chronic CNS diseases, disorders or conditions which may be treated by administration of PKCd inhibitors as described herein. The present methods and compositions can be administered to subjects who are predisposed to or who have progressed into a disease, disorder or condition associated with neuroinflammation. These disorders are diverse and include, for example, acute brain injury (as may result from a surgical procedure performed on the brain), acute spinal cord injury, sepsis, stroke and cerebral ischaemia, epilepsy, neural tube defects in the embryonic development, viral encephalitis, cerebrovascular accidents or cranial trauma, multiple sclerosis, Experimental autoimmune encephalomyelitis (EAE), experimental autoimmune neuritis (EAN), Guillain Barre Syndrome, motor neuron disease, movement disorders, disorders of related systems of the retina and of muscle, including optic neuritis, macular degeneration, diabetic retinopathy, and dermatomyositis, Alzheimer's disease (AD), Parkinson's disease, Huntington's disease, Guillain Barre syndrome, myasthenia gravis, amyotropic lateral sclerosis, Creutzfeldt-Jakob disease, progressing motor neuron disease, depression, anxiety, bipolar disorder, and schizophrenia, HW-1-associated dementia (HAD), neuro-AIDS, Mild Cognitive Impairment, prion disease, minor cognitive/motor dysfunction, head trauma, cerebral amyloid angiopathy, prion diseases, meningitis, myelin degradation, Down's syndrome, post-ischemic brain injury, encephalopathy, senile dementia, amyotrophic lateral sclerosis, and certain disorders involving the peripheral nervous system, such as myasthenia gravis and Duchenne's muscular dystrophy. See, Lucas et al, Br. J. Pharmacol. I47:S232-S240, 2006). According to the invention, a PKCd may be administered to a mammal for the treatment or prevention of neuroinflammation. It is contemplated that the PKCd inhibitor may be administered to a subject at risk for developing neuroinflammation. Accordingly, the PKCd inhibitor may be administered prior to inflammation of the CNS, prior to over-activation of the microglial cells, prior to the onset of oxidative-stress, or prior to neurodegeneration or combinations thereof. See Qin et al. (2007) Glia. 55:453-462.

In another embodiment, a method for the treatment of a neuroinflammatory disease, disorder or condition involving demyelinization is provided. The method includes administering to a mammal suffering from or risk for a neuroinflammatory disease, disorder or condition involving demyelinization, including without limitation those involving myelin degradation, for example multiple sclerosis Multiple sclerosis (MS) Experimental autoimmune encephalomyelitis (EAE), experimental autoimmune neuritis (EAN), or Guillain Barre Syndromean and the like an effective amount of a PKCd inhibitor.

In another embodiment, a method for treating, inhibiting or preventing amyloid fibril formation, inhibit or prevent amyloid fibril growth, and/or cause disassembly, disruption, and/or disaggregation of pre-formed amyloid fibrils and amyloid protein deposits is provided. In one aspect, the method includes administering to a mammal an effective amount of a PKCd inhibitor. In another aspect, the method includes contacting a microglial cell with an effective amount of a PKCd inhibitor. Activity can be measured in vitro by methods such as those described in U.S. Pat. No. 7,514,583, while activity of the PKCd inhibitor in vivo against amyloid diseases can be measured in animal models, such as those APP transgenic mouse models that mimic many of the neuropathological hallmarks of Alzheimer's disease, and in humans.

“Amyloid diseases” or “amyloidoses” suitable for treatment with the PKCd inhibitors are diseases associated with the formation, deposition, accumulation, or persistence of amyloid fibrils, especially the fibrils of an amyloid protein selected from the group consisting of Abeta amyloid, AA amyloid, AL amyloid, IAPP amyloid, PrP amyloid, alpha2-microglobulin amyloid, transthyretin, prealbumin, and procalcitonin, especially Abeta amyloid and IAPP amyloid. Suitable such diseases include Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral beta-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, the amyloidosis of the prion diseases, Creutzfeldt-Jakob disease, Gerstmann-Straussler syndrome, kuru, scrapie, the amyloidosis associated with carpal tunnel syndrome, senile cardiac amyloidosis, familial amyloidotic polyneuropathy, and the amyloidosis associated with endocrine tumors, especially Alzheimer's disease and type 2 diabetes and the like. In one aspect, the method includes treating an “amyloid diseases” or “amyloidoses” by administering to a mammal an effective amount of a PKCd inhibitor. In another aspect, the method includes contacting a microglial cell with an effective amount of a PKCd inhibitor.

PKCd inhibitors may also be used to treat, inhibit or prevent alpha-synuclein/NAC fibril formation, inhibit or prevent alpha-synuclein/NAC fibril growth, and/or cause disassembly, disruption, and/or disaggregation of preformed alpha-synuclein/NAC fibrils and alpha-synuclein/NAC-associated protein deposits in vitro, ex vivo or in vitro. Their activity can be measured in vitro by methods such as those discussed in U.S. Pat. No. 7,514,583, or in vivo in animal models, such as those alpha-synuclein transgenic mouse models that mimic some of the neuropathological hallmarks of Parkinson's disease, and in humans. In one aspect, the method includes administering to a mammal in need thereof an effective amount of a PKCd inhibitor. In another aspect, the method includes contacting a microglial cell with an effective amount of a PKCd inhibitor.

“Synuclein diseases” or “synucleinopathies” suitable for treatment with the PKCd inhibitors are diseases associated with the formation, deposition, accumulation, or persistence of synuclein fibrils, especially alpha-synuclein fibrils. Suitable such diseases include Parkinson's disease, familial Parkinson's disease, Lewy body disease, the Lewy body variant of Alzheimer's disease, dementia with Lewy bodies, multiple system atrophy, and the Parkinsonism-dementia complex of Guam and the like. In one aspect, the method includes treating “synuclein diseases” or “synucleinopathies” by administering to a mammal in need thereof an effective amount of a PKCd inhibitor. In another aspect, the method includes contacting a microglial cell with an effective amount of a PKCd inhibitor.

As mentioned elsewhere herein, PKCd inhibitors and methods of this invention can be used to inhibit the activity of or decrease levels of neurotoxins, such as superoxide and/or hydrogen peroxide production. In one aspect, PKCd inhibitors decrease or inhibit the level of superoxide, nitric oxide and/or hydrogen peroxide produced within and/or released from microglial cells or other immune cells in the CNS. In one aspect the method includes inhibiting or decreasing the level or activity of NFkB, IL-6, and/or an NADPH oxidase (Nox) enzyme. The Nox may be any member of the NADPH oxidase enzyme family such as Nox1, Nox2, Nox3, Nox4 or Nox5 enzyme or isoforms of NADPH or combinations thereof. Accordingly, the invention provides compositions and methods to decrease NFkB, IL-6 and/or Nox enzyme levels and/or activity, or to decrease superoxide and/or hydrogen peroxide levels in the CNS, to treat patients, e.g. mammals, with psychosis, schizophrenia, dementing disorders, CNS inflammation, delirium, depression, traumatic war neurosis, post traumatic stress disorder (PTSD) or post-traumatic stress syndrome (PTSS, Amyotrophic Lateral Sclerosis (ALS, or Lou Gehrig's Disease), Multiple Sclerosis (MS), and cognitive, learning or memory impairments resulting therefrom and the like. See WO/2009/052454.

PKCd inhibitors and methods of this invention can be used to suppress microglial activation, The invention provides compositions and methods to decrease or inhibit microglial production and/or release of a prostaglandin, a leukotriene, and/or thromboxane in the CNS, Exemplary prostaglandins, leukotrienes, and thromboxanes are described elsewhere herein as well as being known to one skilled in the art, The invention is also useful to treat patients, e.g. mammals, with psychosis, schizophrenia, and other diseases, disorders or conditions associated with, caused by or related to microglial production and/or release of any prostaglandin, leukotriene, or thromboxane.

In some circumstances, such as for the treatment of brain tumors, it may be desirable to increase the level of pro-inflammatory substances in the CNS. Accordingly, a method of increasing levels of proinflammatory substances produced within or released from a microglical cell is provided. The method includes contacting a microglical cell, e.g. an activated microglial cell, with a PKCd activator, that is a compound, that increases PKCd expression levels or activity, is provided. In some cases, the PKCd activator is PKCd mRNA or protein.

The skilled artisan can readily perform the in vivo tests to determine the route of administration of the PKCd inhibitor, the amount or dose of PKCd inhibitor to administer, the formulation of the PKCd inhibitor, and the time at which neuroinflammation and levels of pro-inflammatory substances should be assessed.

The PKCd inhibitors may be administered to any region that will decrease or prevent inflammation of the CNS. For example, sites that the PKCd inhibitors may be administered to include but are not limited to the optic nerve, retina, the spinal cord, the brain, including without limitation the forebrain including but not limited to the cerebrum, thalamus, basal ganglia and hypothalamus, the midbrain including but not limited to the tectum and tegmentum, substantia nigra, the hindbrain including but not limited to the cerebellum, pons and medulla, striatum, the frontal cortex, nucleus accumbens, hypothalamus, amygdala, hippocampus, hypothalamus, adrenal gland, or brain stem or combinations thereof. In some cases, the PKCd may be delivered outside of the CNS, for example, intranasally, but will be able to cross or by pass the blood brain barrier. The intranasal administration of peptides to treat CNS conditions is known in the art (see, e.g., U.S. Pat. No. 5,567,682 to Pert, regarding intranasal administration of peptide T to treat AD). Preparation of a PKCdelta inhibitor for intranasal administration may be carried out using techniques as are known in the art.

In particular, the present invention contemplates administering PKCd inhibitors prior to over-activation of the microglial cells, prior to the onset of oxidative-stress, prior to neuroinflammation, or prior to neurodegeneration or combinations thereof. PKCdelta inhibitors may be administered acutely (i.e., prior to, during the onset or shortly after events leading to cerebral inflammation or ischemia), or may be administered prophylactically (e.g., before scheduled surgery, or before the appearance of neurologic signs or symptoms, or to a person at risk for developing a condition), or administered during the course of a degenerative disease to reduce or ameliorate the progression of symptoms that would otherwise occur. The timing and interval of administration is varied according to the subject's symptoms, and may be administered at an interval of several hours to several days, over a time course of hours, days, weeks or longer, as would be determined by one skilled in the art.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral vectors, including retrovirus, adenovirus, adeno-associated virus, herpesvirus and poxvirus, and viral coat protein-liposome mediated transfection (Dzau et al., (1993) Trends in Biotechnology 11. 205-210; Sandmair A M, et al., (2000) Adv. Exp. Med. Biol. 465:423-429; Walther W et al., (2000) Drugs 60(2):249-271). Viral delivery of nucleic acids to targets for gene therapy has become standard practice in the art. Direct administration of bare nucleic acid is also possible.

PKCd inhibitors can be delivered intracellularly using a lipid-mediated protein delivery system. Kaul S, Kanthasamy A, Kitazawa M, Anantharam V, Kanthasamy A G, Caspase-3 dependent proteolytic activation of protein kinase C delta mediates and regulates 1-methyl-4-phenylpyridinium (MPP⁺)-induced apoptotic cell death in dopaminergic cells. relevance to oxidative stress in dopaminergic degeneration. Eur J Neurosci. 2003 September; 18(6):1387-401. Other exemplary techniques and methods for delivering and administering PKCd inhibitors to cells are described elsewhere herein and one skilled in the art will be familiar with such techniques.

The blood-brain barrier presents a barrier to the passive diffusion of substances from the bloodstream into various regions of the CNS. However, active transport of certain agents is known to occur in either direction across the blood-brain barrier. Substances that may have limited access to the brain from the bloodstream can be injected directly into the cerebrospinal fluid. Cerebral ischemia and inflammation are also known to modify the blood-brain barrier and result in increased access to substances in the bloodstream.

Administration of a compound directly to the brain is known in the art. Intrathecal injection administers agents directly to the brain ventricles and the spinal fluid. Surgically-implantable infusion pumps are available to provide sustained administration of agents directly into the spinal fluid. Lumbar puncture with injection of a pharmaceutical compound into the cerebrospinal fluid (“spinal injection”) is known in the art, and may be used for administration of the PKCdelta inhibitors.

Pharmacologic-based procedures are also known in the art for circumventing the blood brain barrier, including the conversion of hydrophilic compounds into lipid-soluble drugs. The active agent may be encapsulated in a lipid vesicle or liposome. See, for example, U.S. Pat. No. 5,686,416. Intravenous or intraperitoneal administration may also be used to administer the compounds of the present invention.

One method of transporting an active agent across the blood-brain barrier is to couple or conjugate the active agent to a second molecule (a “carrier”), which is a peptide or non-proteinaceous moiety selected for its ability to penetrate the blood-brain barrier and transport the active agent across the blood-brain barrier. Examples of suitable carriers include pyridinium, fatty acids, inositol, cholesterol, and glucose derivatives. The carrier may be a compound which enters the brain through a specific transport system in brain endothelial cells. Chimeric peptides adapted for delivering neuropharmaceutical agents into the brain by receptor-mediated transcytosis through the blood-brain barrier are disclosed in U.S. Pat. No. 4,902,505 to Pardridge et al. These chimeric peptides comprise a pharmaceutical agent conjugated with a transportable peptide capable of crossing the blood-brain barrier by transcytosis. Specific transportable peptides disclosed by Pardridge et al. include histone, insulin, transferrin, and others. Conjugates of a compound with a carrier molecule, to cross the blood-brain barrier, are also disclosed in U.S. Pat. No. 5,604,198 to Poduslo et al. Specific carrier molecules disclosed include hemoglobin, lysozyme, cytochrome c, ceruloplasmin, calmodulin, ubiquitin and substance P. See also U.S. Pat. No. 5,017,566 to Bodor.

An alternative method of administering PKCdelta inhibitors that are peptides is administering to the subject a vector carrying a nucleic acid sequence encoding the peptide, where the vector is capable of entering brain cells so that the peptide is expressed and secreted, and is thus available to microglial cells. Suitable vectors are typically viral vectors, including DNA viruses, RNA viruses, and retroviruses. Techniques for utilizing vector deliver systems and carrying out gene therapy are known in the art. Herpesvirus vectors are a particular type of vector that may be employed in administering compounds of the present invention.

The rAAV vectors can produce long-term, chronic production of an anti-neuroinflammatory agent and may, therefore, provide long-term benefit following a single administration (which may, of course, be repeated as necessary). The anti-neuroinflammatory agents of the present invention, e.g., PKCd inhibitors, can be administered by any of the following methods, with the stipulations provided otherwise herein, to treat the diseases and disorders describe herein. Subcutaneous, intravenous, intrathecal, intramuscular, intranasal, oral, transepidermal, parenteral, by inhalation, intracerebroventricular, or intraparenchymal. A preferred embodiment of administration is intraparenchymal injection. The intraparenchymal injection can be carried out to the exclusion of intrathecal and intracerebroventricular injections.

Recombinant adeno-associated virus vector. The anti-neuroinflammatory agents of the present invention, e.g., PKCd inhibitors, can be administered via an rAAV vector e.g., rAAV2. AAV is a replication-deficient parvovirus native to humans and nonhuman primates that exists in nature in over 100 distinct variants. Many of the AAV serotypes have distinct cell and tissue affinities. AAV has a number of properties that support its potential role in gene therapy, for example, it is generally non-pathogenic, capable of persistent infection, and generally elicits mild innate cytokine response. In addition, its genome is readily modified in proviral plasmids, and recombinant production and purification methods are already in place (see, e.g., Flotte Pediatric Res. 58:1143-1147, 2005).

Adeno-associated virus (AAV) is an integrating human DNA parvovirus which has been proposed for use as a gene delivery vehicle for somatic gene therapy. This small non-enveloped virus contains a 4.6 kb single stranded DNA genome that encodes sets of regulatory and capsid genes called rep and cap. Rep polypeptides (rep78, rep68, rep62 and rep40) are involved in replication, rescue and integration of the AAV genome. The cap proteins (VP1, VP2 and VP3) form the virion capsid. Flanking the rep and cap open reading frames at the 5′ and 3′ ends are 145 by inverted terminal repeats (ITRs), the first 125 by of which are capable of forming Y- or T-shaped duplex structures.

The life cycle of AAV is characterized by both lytic and latent components (B. J. Carter, in Handbook of Parvoviruses, ed., P. Tijsser, CRC Press, pp. 155-168, 1990). During a latent infection, AAV virions enter a cell as an encapsidated single stranded (ss) DNA, and shortly thereafter are delivered to the nucleus where the AAV DNA stably integrates into a host chromosome without the apparent need for host cell division. In the absence of helper virus, the integrated ss DNA AAV genome remains latent but capable of being activated and rescued. The lytic phase of the life cycle begins when a cell harboring an AAV pro virus is challenged with a secondary infection by a herpesvirus or adenovirus which encodes helper functions that are recruited by AAV to aid in its excision from host chromatin (Carter, supra). The infecting parental ssDNA is expanded to duplex replicating form (RF) DNAs in a rep dependent manner. The rescued AAV genomes are packaged into preformed protein capsids (icosahedral symmetry approximately 20 nm in diameter) and released as infectious virions that have packaged either + or − ss DNA genomes following cell lysis.

Recombinant forms of AAV (rAAV) have been developed as vectors by replacing all viral open reading frames with a therapeutic minigene, while retaining the necessary cis elements contained in the ITRs. (see, e.g., U.S. Pat. Nos. 4,797,368; 5,153,414; 5,139,941; 5,252,479; and 5,354,678; and International Publication Nos. WO91/18088 published Nov. 28, 1991; WO93/24641 published Dec. 9, 1993 and WO94/13788 published Jun. 23, 1994). See also U.S. Pat. Nos. 5,756,283; 7,282,199; 7,241,447; and 7,208,315. Transduction with rAAV has been demonstrated in a wide variety of cell types including differentiated, non-dividing cells, suggesting the potential of this vector system for in vivo gene delivery to organs such as muscle, liver, central nervous system and lung.

A PKCd inhibitor or pharmaceutical composition comprising a PKCd may be administered to an individual by various routes including, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, intramuscularly, intraperitoneally, subcutaneously, subconjunctival, intravesicularly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 18th Ed. Mack Printing Company, 1990, incorporated herein by reference). Thus, the for example, the PKCd may be administered orally or parenterally, such as intravenously, intramuscularly, subcutaneously, intraorbitally, intracapsularly, intraperitoneally, intrarectally intracisternally or by passive or facilitated absorption through the skin using, for example, a skin patch or transdermal iontophoresis, respectively. Furthermore, a composition comprising a PKCd inhibitor can be administered by injection, intubation, orally or topically, the latter of which can be passive, for example, by direct application of an ointment or powder, or active, for example, using a nasal spray or inhalant. A PKCd inhibitor also can be administered as a topical spray or an inhalant, in which case one component of the composition is an appropriate propellant. Methods of administering a PKCd inhibitor of the invention include, but are not limited to, parenteral administration (e.g., intradermal, intramuscular, intraperitoneal, intravenous and subcutaneous), epidural, and mucosal (e.g., intranasal and oral routes). The PKCd inhibitor may be administered by any convenient route, for example by infusion or bolus injection, by absorption through epithelial or mucocutaneous linings (e.g., oral mucosa, rectal and intestinal mucosa, etc.) and may be administered together with other agents suitable for treatment. Administration can be systemic or local. The site or region for administration may be selected based on considerations such as ease and efficacy. In one embodiment, it may be desirable to administer the PKCd inhibitors locally to the area in need of treatment; this may be achieved by, for example, and not by way of limitation, local infusion, by injection, or by means of an implant. In yet another embodiment, the PKCd inhibitor can be delivered in a controlled release or sustained release system. In one embodiment, a pump may be used to achieve controlled or sustained release.

The dosage administered will, of course, vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active ingredient {e.g., an Anti-neuroinflammatoryagent) can be about 0.01 to 100 milligrams per kilogram of body weight. Ordinarily 1.0 to 5, and preferably 1 to 10 milligrams per kilogram per day given in divided doses 1 to 6 times a day or in sustained release form is effective to obtain desired results.

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

For example, the dosage of the composition which will be effective in the treatment, prevention or management of a disease can be determined by administering the composition to an animal model such as, for example, the animal models disclosed herein or known to those skilled in the art. In addition, in vitro assays may optionally be employed to help identify optimal dosage ranges. Selection of the preferred effective dose can be determined (e.g., via clinical trials) by a skilled artisan based upon the consideration of several factors which will be known to one of ordinary skill in the art. Such factors may include the disease to be treated or prevented, the symptoms involved, the patient's body mass, the patient's immune status and other factors known by the skilled artisan to reflect the accuracy of administered pharmaceutical compositions. The precise dose to be employed in the formulation will also depend on the route of administration, and the progression of the disease, and should be decided according to the judgment of the practitioner and each patient's circumstances. Effective doses may be extrapolated from dose-response curves derived from in vitro or animal model test systems.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a concentration range that includes the IC₅₀ (i.e., the concentration of the test compound that achieves a half-maximal inhibition of symptoms) as determined in cell culture. Such information can be used to more accurately determine useful doses in humans. Levels in plasma and cerebral spinal fluid may be measured, for example, by high performance liquid chromatography. The protocols and compositions of the invention are preferably tested in vitro, and then in vivo, for the desired therapeutic or prophylactic activity, prior to use in humans. For example, in vitro assays may be used to determine whether administration of a specific therapeutic protocol is indicated.

PKCd inhibitors for use in decreasing levels of pro-inflammatory substances for protection from neuroinflammation can be tested in suitable animal model systems prior to testing in humans, including principle animal models known in the art and widely used. For example, LPS, intranigral injections of TNF-alpha and other pro-inflammatory cytokines and autoimmune disease models, alpha-synuclein overexpression models are suitable mouse models. Also included are Parkinson's disease models such as MPTP, 6-OHDA, paraquat-induced, proteosomal inhibitor-induced, and genetic models, including α-Synuclein, Parkin (PARK 2), MitoPark models.

Optimal dose may be empirically determined. Animals can be sacrificed by focused microwave beam irradiation, for example. Striatal tissue can then be dissected and homogenates can be subjected to immunoblot analysis. The potential efficacy of these PKCd inhibitors in relieving neuroinflammatory related pathological symptoms or in neuroinflammation may be assessed in animal models for disease. For example, treatment of rats or mice with LPS induces inflammation in the brain. Generally, at least two groups of animals are used in the assay, with at least one group being a control group which is administered the administration vehicle without the known PKCd inhibitor or potential PKCd inhibitor.

Anti-neuroinflammatory agents, e.g., PKCd inhibitors, can be administered either as individual therapeutic agents or in combination with one another and/or other therapeutic agents and/or therapeutic regimes. Proteinaceous inhibitors can be administered as proteins or their delivery can be facilitated by any suitable vector construct such as a plasmid or rAAV vector.

Pharmaceutical carriers can be selected on the basis of the chosen route of administration and standard pharmaceutical practice. In one aspect, pharmaceutically acceptable carrier includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water may be a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. The PKCd inhibitors of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The potential PKCd inhibitors can be administered by a variety of ways as discussed previously and in various formulations including as a pharmaceutical composition as discussed below. Routes and formulations are known to one skilled in the art.

In this regard, the PKCdelta inhibitors and other compounds of the invention may be used alone or in combination with other known anti-inflammatory drugs or cytokines to formulate pharmaceutical compositions for the treatment of CNS inflammatory conditions.

In another embodiment, the invention includes pharmaceutical compositions for decreasing levels of pro-inflammatory substances in a central nervous system of a mammal in need thereof. In another aspect, the pharmaceutical compositions may be used for the treatment of a disease, disorder or condition in subjects who are predisposed to or who have progressed into a disease, disorder or condition associated with neuroinflammation. These disorders are diverse and include, for example, acute brain injury (as may result from a surgical procedure performed on the brain), acute spinal cord injury, stroke and cerebral ischaemia, epilepsy, neural tube formation in embryonic development, viral encephalitis, cerebrovascular accidents or cranial trauma, subarachanoid hemmorhage, multiple sclerosis, Experimental autoimmune encephalomyelitis (EAE), experimental autoimmune neuritis (EAN), Guillain Barre Syndrome, motor neuron disease, movement disorders, disorders of related systems of the retina and of muscle, including optic neuritis, macular degeneration, diabetic retinopathy, and dermatomyositis, Alzheimer's disease (AD), Parkinson's disease, Huntington's disease, Guillain Barre syndrome, myasthenia gravis, amyotropic lateral sclerosis, Creutzfeldt-Jakob disease, progressing motor neuron disease, depression, anxiety, bipolar disorder, and schizophrenia, HIV-1-associated dementia (HAD), neuro-AIDS, Mild Cognitive Impairment, prion disease, minor cognitive/motor dysfunction, head trauma, cerebral amyloid angiopathy, prion diseases, meningitis, myelin degradation, Down's syndrome, post-ischemic brain injury, encephalopathy, senile dementia, amyotrophic lateral sclerosis, and certain disorders involving the peripheral nervous system, such as myasthenia gravis and Duchenne's muscular dystrophy. See, Lucas et al, Br. J. Pharmacol. I47:S232-S240, 2006).

In another embodiment, pharmaceutical composition comprising the PKCd inhibitor comprises a PKCd inhibitor in an amount effective for treating neuroinflammation, such as any one of those diseases, disorders, and condition associated with neuroinflammation described elsewhere herein or known to one skilled in the art.

According to the invention, the pharmaceutical composition comprising PKCd may be administered to a mammal for the treatment or prevention of neuroinflammation. It is contemplated that the pharmaceutical composition comprising the PKCd inhibitor may be administered to a subject or population at risk for developing neuroinflammation.

Accordingly, the pharmaceutical composition comprising the PKCd inhibitor may be administered prior to inflammation of the CNS or the onset of neurodegeneration. The pharmaceutical composition may include any suitable PKCd inhibitor, including but not limited to a polynucleotide, peptide, polysaccharide, lipid, small molecule or drug or derivatives thereof. In another aspect, the PKCd inhibitor is (3-[(8-Cinnamoyl-5,7-dihydroxy-2,2-dimethyl-2H-1-benzopyran-6-yl)methyl]-2′,4′,6′-trihydroxy-5-methylaceophenone (rottlerin) or derivatives thereof. In another aspect, the PKCd inhibitor is a siRNA molecule. While various sequences of PKCd siRNAs that may be used in methods and compositions of the present invention are described above, PKCd siRNA molecules are not intended to be limited to these specific disclosed examples. In another aspect, the PKCd inhibitor is a peptide comprising the sequence of Asp Ile Pro Asp. In another aspect, the PKCd inhibitor is dominant negative PKCd mutant of normal, wild type PKCd. The compositions may comprise a therapeutically effective amount of a PKCd inhibitor and a pharmaceutically acceptable carrier.

Pharmaceutical compositions for use in accordance with the present invention may be formulated in using pharmaceutically acceptable carriers or excipients. As discussed above, various delivery systems are known and can be used to administer the PKCd inhibitors of the present invention. As discussed above, the amount of the PKCd inhibitors which will be effective in the treatment, prevention or management of diseases, disorders or conditions described herein can be determined by standard research techniques.

The present invention provides kits that can be used in the above methods. In one embodiment, a kit comprises at least one PKCd inhibitor, in one or more containers, useful for the treatment of a disorder, disease, or condition associated with neuroinflammation or the pro-inflammatory substances released from activated microglial cells. Optionally associated with such container(s) can be a notice in the form prescribed by a governmental agency regulating the manufacture, use or sale of pharmaceuticals or biological products, which notice reflects approval by the agency of manufacture, use or sale for human administration.

All publications, patents and patent applications identified herein are incorporated by reference, as though set forth herein in full. The invention being thus described, it will be apparent to those skilled in the art that the same may be varied in many ways without departing from the spirit and scope of the invention. Such variations are included within the scope of the following claims.

The invention is further illustrated by the following specific examples which are not intended in any way to limit the scope of the invention.

The examples which follow are set forth to illustrate the present invention, and are not to be construed as limiting thereof.

### EXAMPLES

### Role of PKCδ in Regulating Proinflammatory Events in Microglial Cells

### Example 1

### LPS Induced TNFα Release in BV-2 Microglial Cells

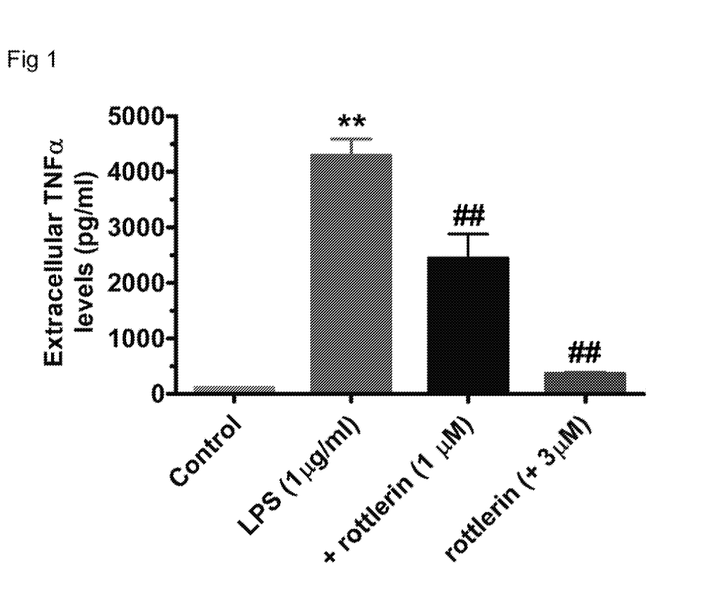

Microglial cells are the primary immune cells in the CNS and are the major source of TNFα during neuroinflammation. Therefore, in this section, we examined the possible mechanisms that could regulate TNFα release during microglial activation. In particular, we examined whether PKCδ plays any role in the production and release of cytokines. For these studies, we used LPS as a microglial activator on BV2 cells, a widely used microglial cell line. BV-2 cells were exposed to LPS (1 μg/ml) in the presence or absence of PKCδ inhibitor rottlerin (1-3 μM). At this dose range, rottlerin effectively inhibits PKCδ and does not cause any toxicity [75]. After 16 hr, cell-free supernatants were collected and TNFα levels were quantified using a multiplex bead-based luminex® assay system, as described in the methods. As shown in FIG. 1, LPS treatment induced a dramatic increase (˜8-fold) in the release of TNF and PKCδ inhibitor rottlerin dose-dependently blocked LPS-induced TNFα release, suggesting that PKCδ positively regulates TNFα release during inflammatory response. We also measured IL-6 and IL-12 in LPS and rottlerin treated samples but found no effect of rottlerin on LPS-induced IL-6 and IL-12 levels and a 24 hr exposure to 300 μM MPP failed to release TNFα from BV-2 cells (data not shown).

### Example 2

### PKCδ Mediates LPS Induced Release of TNFα in BV-2 Microglial Cell Line

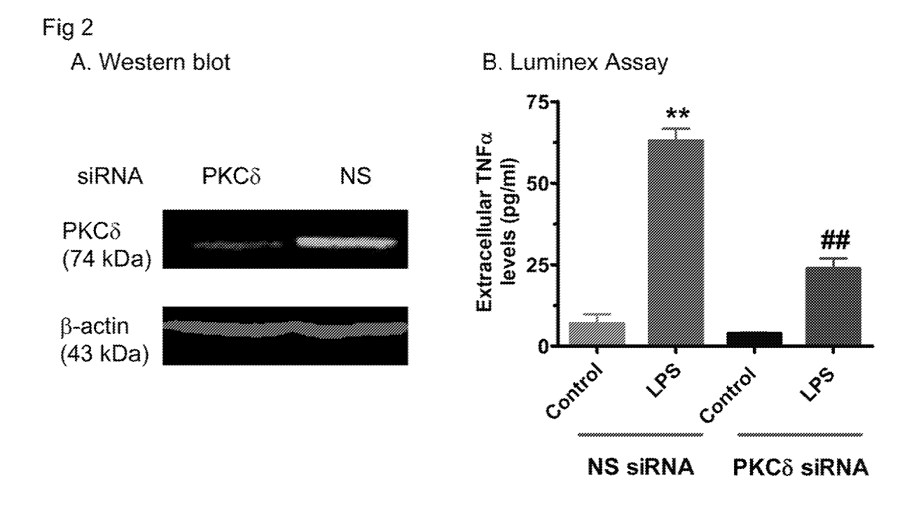

To further validate the interesting results obtained above with the pharmacological PKCδ inhibitor rottlerin, we adopted an RNAi approach. In this experiment, we examined whether PKCδ-siRNA mediated knockdown of PKCδ, would suppress LPS-induced TNFα release in BV-2 cells. FIG. 2A shows the PKCδ protein knockdown in PKCδ-siRNA transfected BV-2 cells but not in cells transfected with non-specific siRNA (NS-siRNA). We observed that PKCδ-siRNA transfection ameliorated the TNFα release from BV-2 cells in response to LPS treatment, as compared to non-specific siRNA transfected cells (FIG. 2B). Together, these data suggest that PKCδ can regulate TNFα release in microglial cells during neuroinflammation.

### Example 3

### LPS-Induced Upregulation and Membrane Translocation of PKCδ in BV-2 Microglial Cells

Since PKCδ regulates TNFα release during neuroinflammatory insults, we aimed to characterize the mode of activation of the kinase in microglial cells. We initially thought that PKCδ could be activated through proteolytic cleavage similar to that of dopaminergic cells. Interestingly, we found no evidence of PKCδ cleavage in BV2 microglial cells, but rather a significant upregulation of the native PKCδ band (74 kDa) in the western blot following 24 hr of LPS treatment (FIG. 3A). We further examined whether subcellular localization of PKCδ is altered in microglial cells during LPS treatment. Control and LPS-treated BV-2 cells were fixed with 4% paraformaldehdye and stained with PKCδ antibody followed by secondary staining with Alexa488-conjugated antibody. Images were captured by Nikon TE200 microscope. Again to our surprise, we found a dramatic translocation of PKCδ to the plasma membrane in LPS treated BV-2 cells as compared to control cells (FIG. 3B). Unlike dopaminergic neuronal cells, no nuclear translocation was observed in BV-2 cells, demonstrating a distinct mode of PKCδ activation in microglia cells vs neuronal cells in response to neuroinflammatory insult. Collectively, the upregulation and membrane translocation of PKCδ in BV-2 cells during LPS stimulation suggest that increased expression and membrane translocation may contribute to sustained release of TNFα during chronic neuroinflammation.

### Example 4

### Proinflammatory Cytokine TNFα Induces Increases in PKCδ Promoter Activity in BV-2 Cells

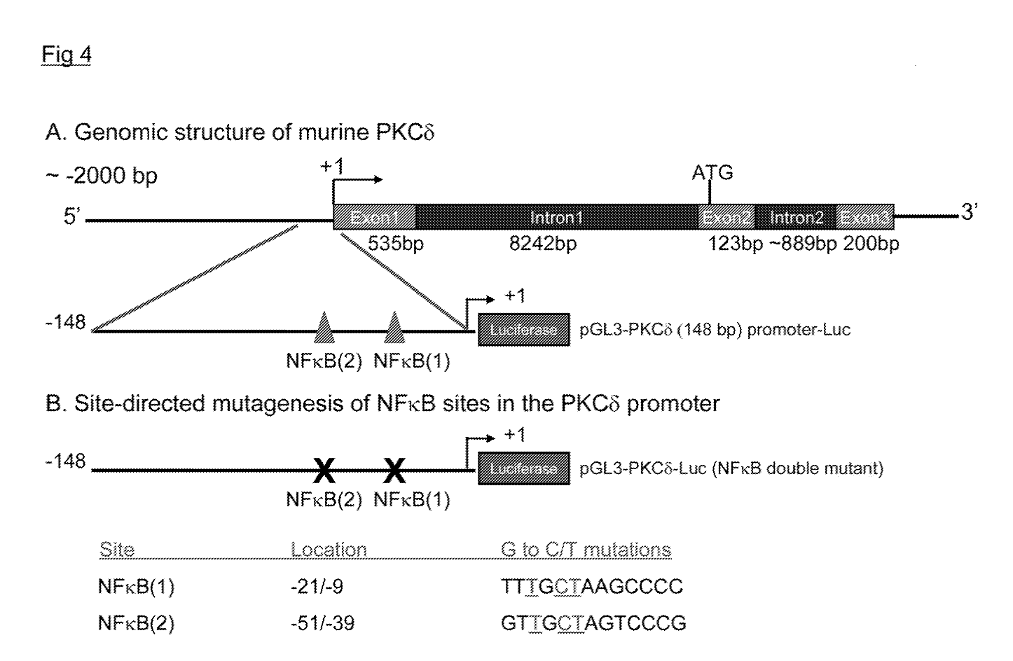

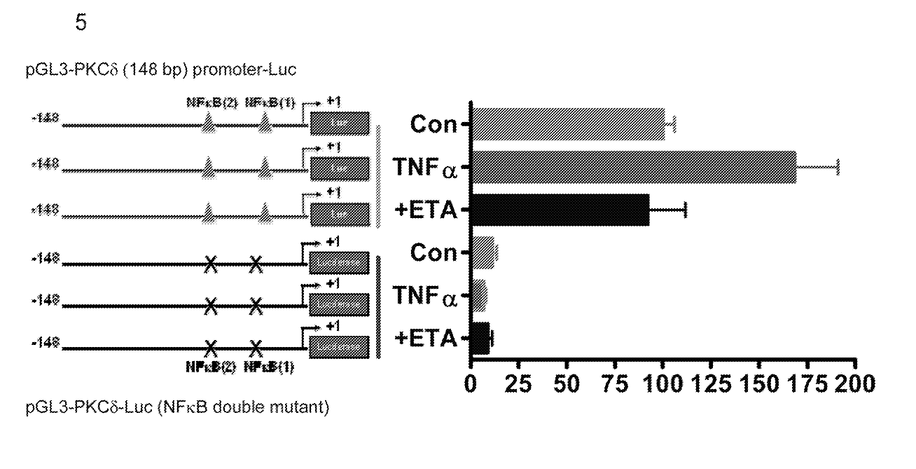

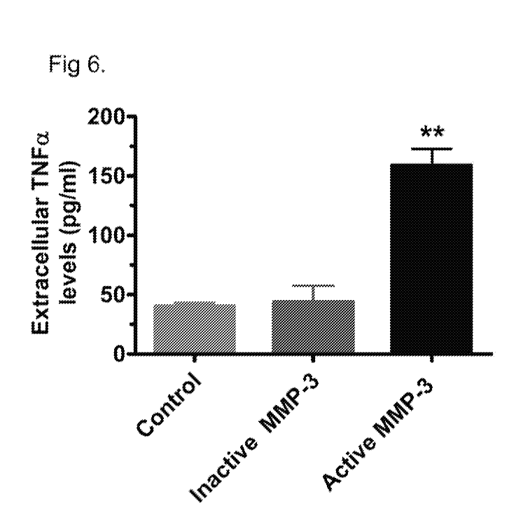

Since PKCδ upregulation was observed during neuroinflammation (FIG. 4A), we sought to determine mechanisms that regulate PKCδ expression via transcriptional activation. We first cloned the full-length mouse PKCδ promoter (2 Kb) from genomic DNA using the fusion PCR technique (FIG. 5A). The resulting PKCδ promoter 2 Kb fragment was cloned into the promoter-less pGL3-luciferase reporter vector. Binding of the transcription factors to the PKCδ promoter region would result in the expression of luciferase enzyme. Bioinformatic analysis revealed that the 148 by PKCδ promoter nearest to the start codon (−1 to −148) is enriched with many transcriptional factor binding sites, including two NFκB binding sites (FIG. 5A). We identified this 148 by PKCδ promoter functions as efficiently as the full-length PKCδ promoter (2 Kb) in luciferase reporter assays (unpublished observations). We focused our attention on the NFκB binding site because of the known proinflammatory role of this transcription factor in immune function. To determine the role of two NFκB sites that are present in the PKCδ promoter, we mutated the two NFκB sites and cloned them into the pGL3-Luciferase vector (FIG. 5B). The plasmids coding for the minimal 148 by PKCδ promoter and minimal promoter with mutated NFκB sites were transfected into BV-2 cells and exposed to the proinflammatory cytokine TNFα. FIG. 6 shows TNFα-induced a significant increase in PKCδ promoter activity in BV-2 cells expressing the 148 by minimal PKCδ promoter construct, whereas TNFα failed to induce PKCδ promoter activity in BV-2 cells expressing the minimal promoter construct with mutated NFκB sites. Etanercept also blocked TNFα-induced PKCδ promoter activity. The basal PKCδ promoter activity was also significantly reduced in NFκB mutated promoter constructs, suggesting the importance of NFκB sites for basal expression. Together, these results indicate that NFκB-transcription sites present in PKCδ promoter are critical for PKCδ upregulation during inflammatory stimulation.

### Example 5

### Active MMP3 Induces the TNFα-Release in BV-2 Cells

Matrix metalloproteinases (MMPs) are a family of highly homologous protein-degrading zinc dependent endopeptidases. MMPs have been shown to play a role in many biological processes, including inflammation. Recent studies have demonstrated that MMP-3 released from apoptotic dopaminergic neuronal cells during neurotoxic insults can activate microglia in the vicinity of the dopaminergic system [44, 102]. Therefore, we examined if MMP-3 could trigger TNFα release in BV-2 cells. In this experiment BV-2 cells were exposed to either inactive proMMP-3 (400 ng/ml) or MMP-3 that was activated using 4-Aminophenylmercuric acetate (APMA); Active MMP-3 (400 ng/ml). Following 24 hr of MMP-3 treatment, supernatants were collected from extracellular medium and TNFα levels were determined using a Luminex immunoassay. FIG. 6 shows that ActMMP-3 induced a 3-fold increase in the levels of TNFα, whereas ProMMP3 treatment did not induce any significant levels of TNFα compared to control BV-2 cells. These results suggest that microglial activation by endogenously released factors such as MMP-3 can also induce TNFα release. In the proposed experiments, we plan to elucidate the PKCδ-dependent signaling mechanisms in neuroinflammatory processes of nigral dopaminergic system using some key inflammatory stimuli including MMP-3, TNFα, LPS and MPP+.

### Example 6

### Isolation and Characterization of Primary Microglia from C57 Black Mice

In the original proposal, we showed that LPS-treatment induces TNFα-release in the BV2 microglial cell line (FIG. 1). To extend the results obtained with the BV2 microglial cell line to primary microglia, we performed some preliminary studies in primary microglia isolated from post-natal day 2 (PND-2) C57 black mice as described previously (Floden et al. 2006). Briefly, the brain tissues from PND-2 mice were gently triturated in ice-cold Ca²⁺ and Mg²⁺-free HBSS. The cells were spun down and cultured using DMEM/F-12 medium containing 10% heat-inactivated FBS, antibiotics and essential nutrients, as described previously (Liu et al. 2002). After 12-14 days, microglia were harvested by the differential shaking method (350 rpm for 4 hr) and grown on poly-D lysine coated 24-well plates. Primary microglia were then fixed with 4% paraformaldehyde and processed for immunohistochemical staining using CD 11b (microglial marker) monoclonal antibody followed by Alexa 568-conjugated secondary antibody (red). Nuclei were stained with TO-PRO-3 dye (blue). The cells were viewed using a Nikon TE2000 microscope, and images were captured by SPOT digital camera. FIG. 1 shows the CD11b-positive microglia in culture, and quantitative analysis of CD11b+ immunostaining revealed that the enriched microglial population was >95% pure. Additionally, we have established a microglia isolation procedure using magnetic labeled CD11b (MACs CD11b+) microbeads followed by AutoMACS cell sorting, which also yielded over 95% pure microglia.

### Example 7

### PKCδ Inhibitor Suppresses LPS-Induced TNFα-Release in Primary Microglia

In the original proposal, we showed that LPS-induced TNFα-release from the BV2 microglial cell line was significantly blocked by pretreatment with the PKCδ inhibitor rottlerin (FIG. 1), suggesting that PKCδ plays a role in TNFα release following neuroinflammatory insult. We extended this study to primary microglia. CD11b+ primary microglia cultures were prepared, as described in the previous section. The primary microglial cultures were treated with 100 ng/ml LPS for 24 hr in the presence of various doses of the PKCδ inhibitor rottlerin (1 μM and 3 μM). TNFα levels in the culture medium were quantified using a multiplex bead-based Luminex® immunoassay. As shown in FIG. 2, LPS induced a dramatic release of TNFα (7085±640 pg/ml) from primary microglia as compared to a very low basal TNFα release (3.2 pg/ml) from untreated microglia. Rottlerin treatment significantly suppressed LPS-induced TNFα levels in a dose dependent manner. 3 μM rottlerin reduced LPS-induced TNFα release by 75%. Consistent with the BV2 cell line data presented in the original proposal, the new results provide further evidence that PKCδ can regulate TNFα release in primary microglia during neuroinflammation.

### Example 8

### Proteolytic Activation of PKCδ in Mouse Substantia Nigra Following Neuroinflammatory Insult

To further extend the cell culture data to animal models, we performed preliminary studies in an animal model of neuroinflammation. The stereotaxic injection model for LPS has recently been used to study the nigral degenerative events associated with neuroinflammation (McCoy et al 2006). C57 black mice were anesthetized and placed in a stereotaxic frame. As depicted in FIG. 3A, LPS (2 μg in a 20 volume) was stereotaxically injected into the right side of the mouse substantia nigra with saline on the left side (stereotaxic coordinates. Bregma AP, −3.2 mm, ML, ±2.0 mm, DV, −4.7 mm). Seven days after LPS injection the mice were sacrificed and the substantia nigra was dissected out. The tissue lysate was subjected to the immunoprecipitation-PKCδ kinase assay using a histone substrate, as described in the methods section of the original proposal. The kinase assay was performed in the absence of lipid cofactors to specifically determine the kinase activity of the proteolytically activated PKCδ as described in our publications (Anantharam et al., 2002). TH staining was used to confirm the accurate dissection of the nigral tissue, and beta-actin was used to ensure equal protein levels. As shown in FIG. 3B, LPS injection significantly increased the PKCδ kinase activity in the mouse substantia nigra. Densitometric analysis of the phosphorylated histone bands revealed that LPS treatment significantly increased kinase activity by 2.5-fold as compared to saline-injected samples. Together, for the first time, we demonstrated the proteolytic activation of PKCδ in the substantia nigra during neuroinflammation.

### Example 9

### Additional Data and Results

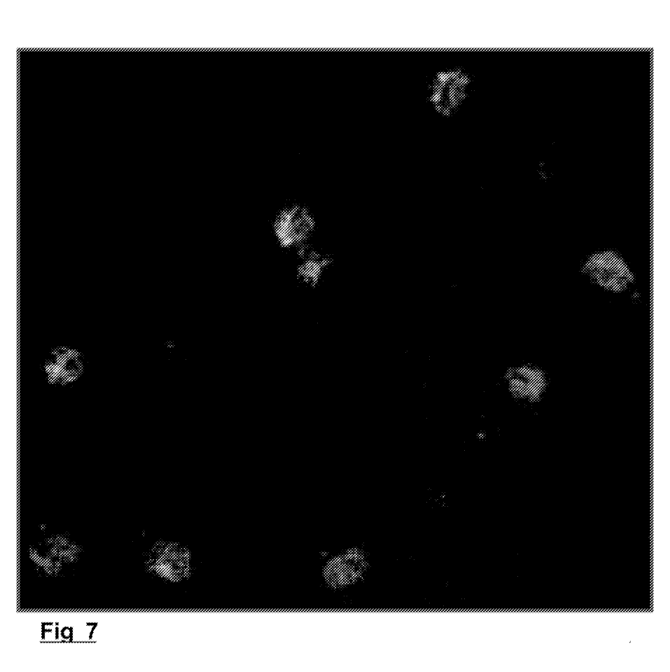

FIG. 7. Immunostaining of primary mouse microglia cells with CD11b+. Primary microglia was isolated from PND2 C57black pups and processed for CD11b immunofluoresence labeling as described in the text.

FIG. 8. Effect of PKCδ inhibitor rottlerin on LPS-induced TNFα release from primary mouse microglia. Primary microglia were treated with 100 ng/ml LPS for 24 hr in the presence of PKCδ inhibitor rottlerin (1 μM and 3 μM). TNFα levels in the culture medium were quantified using a multiplex bead-based luminex® immunoassay. *** p<0.001 compared to controls; ## p<0.05 ### p<0.001 compared to LPS treatment; N=5.

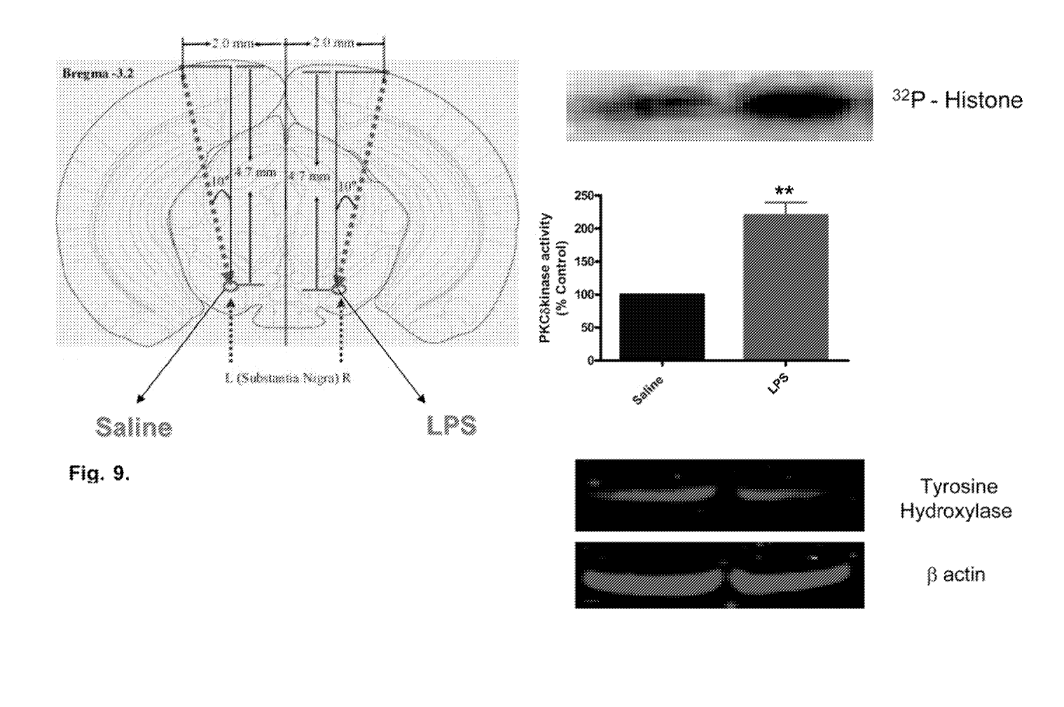

FIG. 9. Intranigral LPS-induced neuroinflammatory animal model. A) Stereotaxic coordinates used in the injection of LPS in the substantia nigra of C57 black mice. B) PKCδ kinase activity measured by the 32P ATP immunoprecipitation kinase assay. **p<0.05compared to saline; N=3.

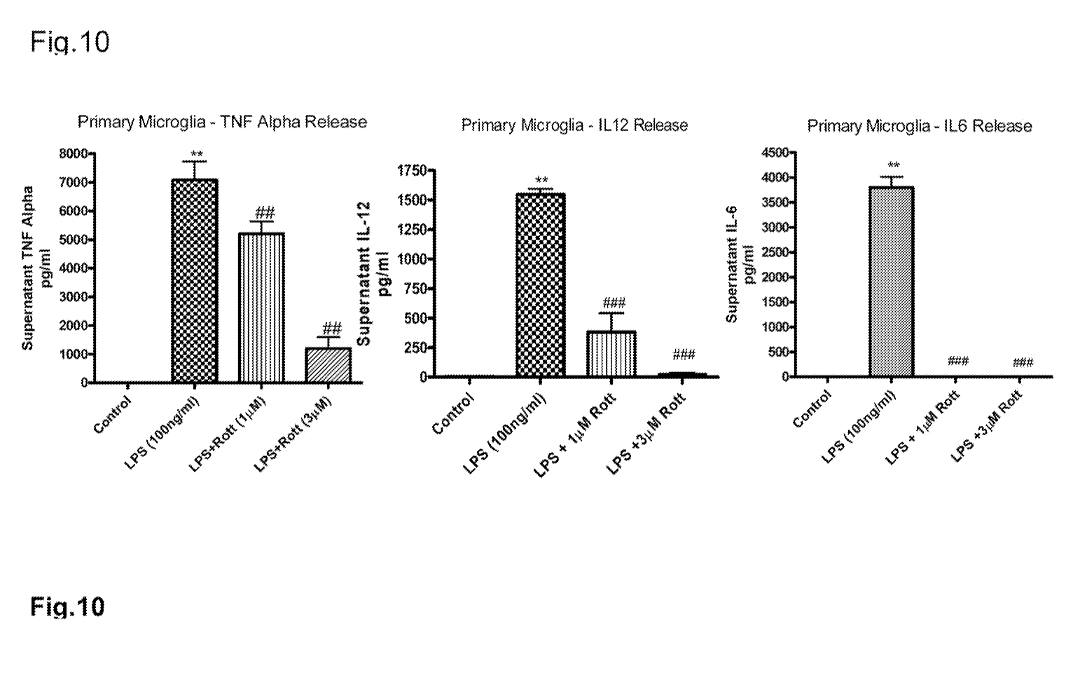

FIG. 10. PKCδ inhibitor rottlerin suppresses pro-inflammatory cytokine release in primary mouse microglia. Primary Microglia were isolated by differential adherence from post-natal mixed-glial cultures and purity verified to be more than 95% by immunocytochemistry. Primary microglia were treated with LPS (100 ng/ml) for 24 hrs or pre-treated with the PKCδ inhibitor rottlerin (1 μM) and then treated with LPS. After treatment, supernatants were collected and proinflammatory cytokine levels were assayed using a multiplexed luminex assay.

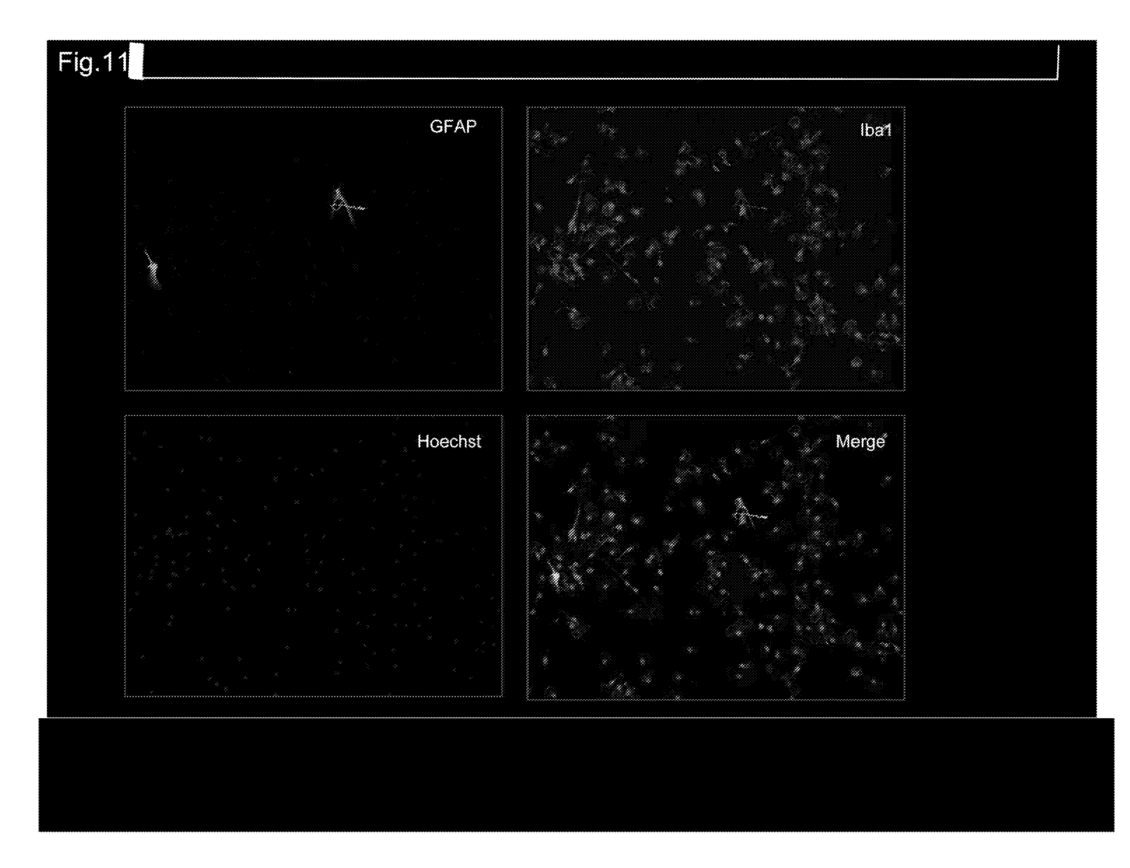

FIG. 11. Purity of Primary Microglial Cultures Assessed by Double Immunofluorescence. Primary microglial cultures obtained by differential adherence were verified to be >95% pure by double immunofluorescence using GFAP as the astrocyte marker and Iba1 as the microglial marker. The nucleus was labeled with Hoechst.

FIG. 12. PKC-delta Lentiviral-shRNA Suppresses LPS-induced Nitric Oxide Release (a marker of proinflammatory effect) in Primary Mouse Microglia. Primary Microglia isolated by differential adherence from post-natal mixed-glial cultures were transduced with either PKC-delta or non-specific shRNA expressing lentivirus and treated with LPS for 24 hrs. Supernatants were collected and the nitric oxide release was determined by measuring the amount of released nitrite using the griess assay. PKC δ shRNA and Control shRNA Lentiviral vector were obtained from Santa Cruz Biotechnology, Santa Cruz, Calif. PKC δ shRNA (m) Lentiviral Particles. sc-36246-V (from Santa Cruz Biotechnology 2008-2009 catalog). PKC δ shRNA (m) Lentiviral Particles are concentrated, transduction-ready viral particles containing a target-specific construct that encodes a 19-25 nt (plus hairpin) shRNA designed to knock down gene expression. Each vial contains 200 μl frozen stock containing 1.0×10⁶ infectious units of virus (IFU) in Dulbecco's Modified Eagle' Medium with 25 mM HEPES pH 7.3. Suitable for 10-20 transductions. Control shRNA Lentiviral Particles. sc-108080 (from Santa Cruz Biotechnology 2008-2009 catalog). Control shRNA Lentiviral Particles is a negative control for experiments using targeted shRNA Lentiviral Particle transduction; Control shRNA Lentiviral Particles encodes a scrambled shRNA sequence that will not lead to the specific degradation of any known cellular mRNA. Each vial contains 200 μl shRNA lentiviral particles sufficient for 10-20 transductions.

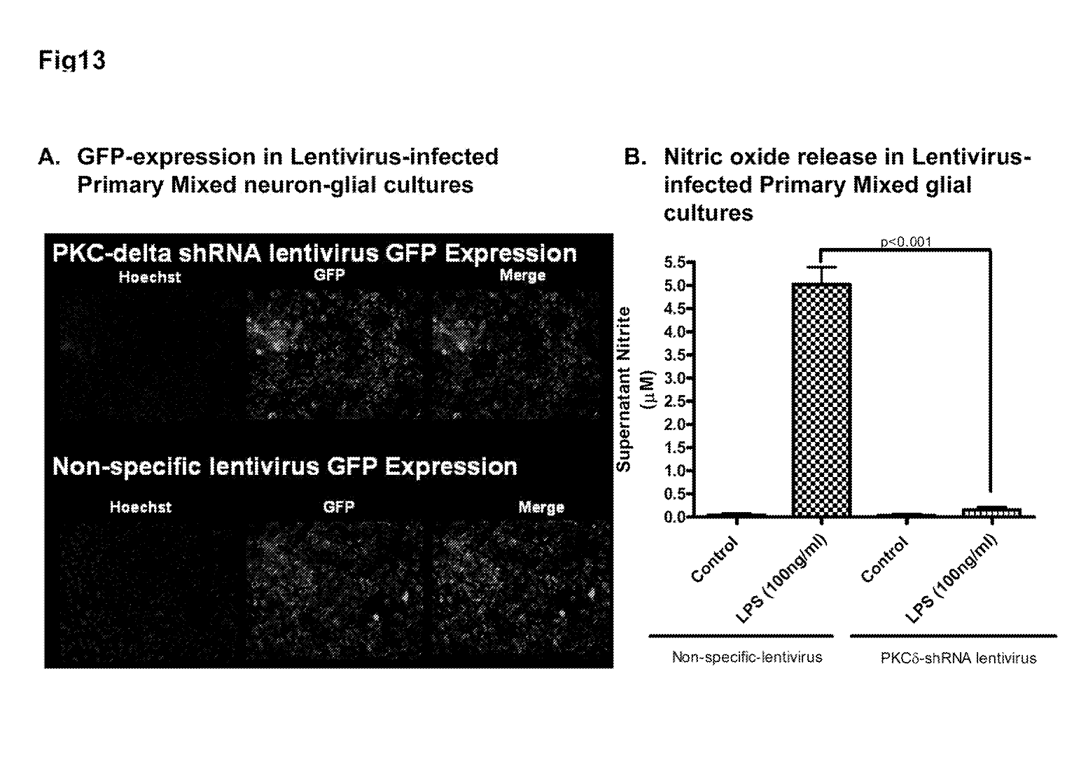

FIG. 13. PKC-delta Lentiviral-shRNA Suppresses LPS-induced Nitric Oxide Release in mouse midbrain neuron-glia cultures. Primary mouse ventral midbrain neuron glial cultures obtained from E14 mouse brain and were transduced with either PKCdelta shRNA or nonspecific shRNA lentivirus at 4 days in vitro (DIV-4) and treated with LPS at DIV10. PKC-delta Lentiviral-shRNA has been described elsewhere herein, for example, in the description of FIG. 12. Supernatants were collected at 48 hrs and nitrite levels were determined using the griess assay.

FIG. 14. Native PKC 8 protein is upregulated during microglial activation and is phosphorylated at its activation loop site Thr 505 (pThr 505) in primary microglial cells. Primary microglia isolated by differential adherence from post-natal mixed-glial cultures were treated with LPS (100 ng/ml) or aggregated alpha synuclein (250 nM) that was aged in vitro for 7 days. PKC-delta and phospho PKC-delta Thr-505 levels were determined by western blotting using specific antibodies.

FIG. 15. Activation loop phosphorylation of PKCδ (pThr 505) is highly upregulated in vivo in the acute MPTP mouse model of Parkinson's Disease. C57BL6 mice were treated were injected with 4 doses of MPTP (18 mg/kg; i.p.) at 2 hr intervals and sacrificed 24 hrs after the last injection. The substantia nigra and striatum were dissected using a brain matrix. Activation loop phosphorylation of PKC-delta was determined using a phospho-specific antibody. Beta actin was used as the loading control and Tyrosine hydroxylase western blotting was used to confirm accurate dissection of the brain regions.

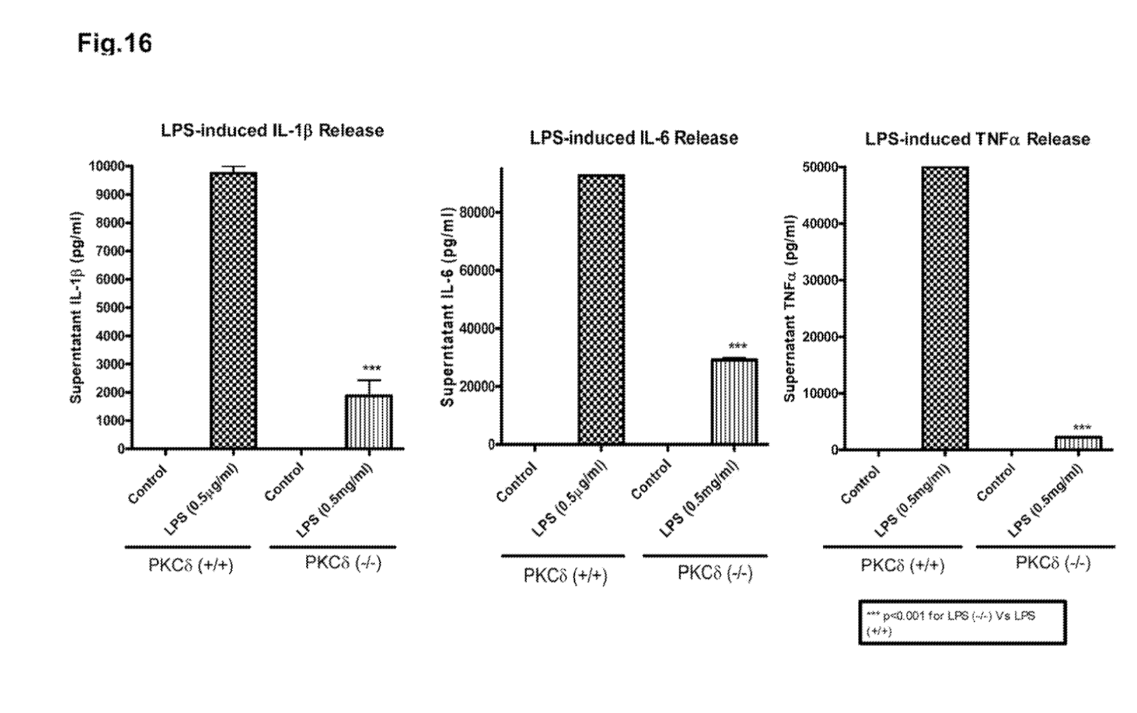

FIG. 16. Primary microglia were isolated from PKC delta knockout (−/−) and wild type (+/+) mice by differential adherence and treated with LPS (0.5 μg/ml) for 24 hr. Proinflammatory cytokines Th-1β, IL-6 and TNFα were determined using a multiplexed luminex immunoassay.

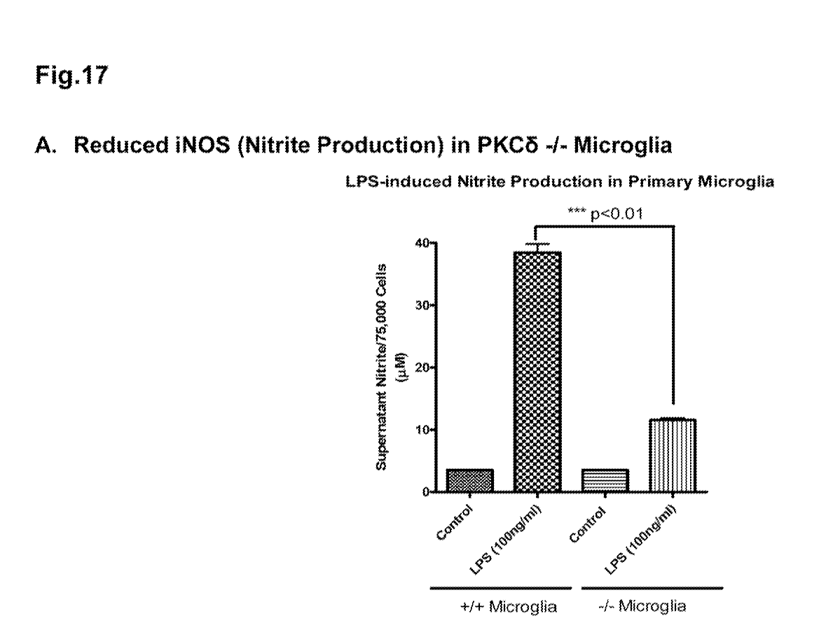

FIG. 17. Primary Microglia were isolated from PKC-delta wild type (PKCδ +/+) and knockout (PKCδ−/−) mice and treated with LPS for 24 hrs. Nitric oxide release was determined by measuring the amount of nitrite using the griess assay and a sodium nitrite standard curve. The results indicate that Reduced iNOS (Nitrite Production) in PKCδ −/− Microglia.

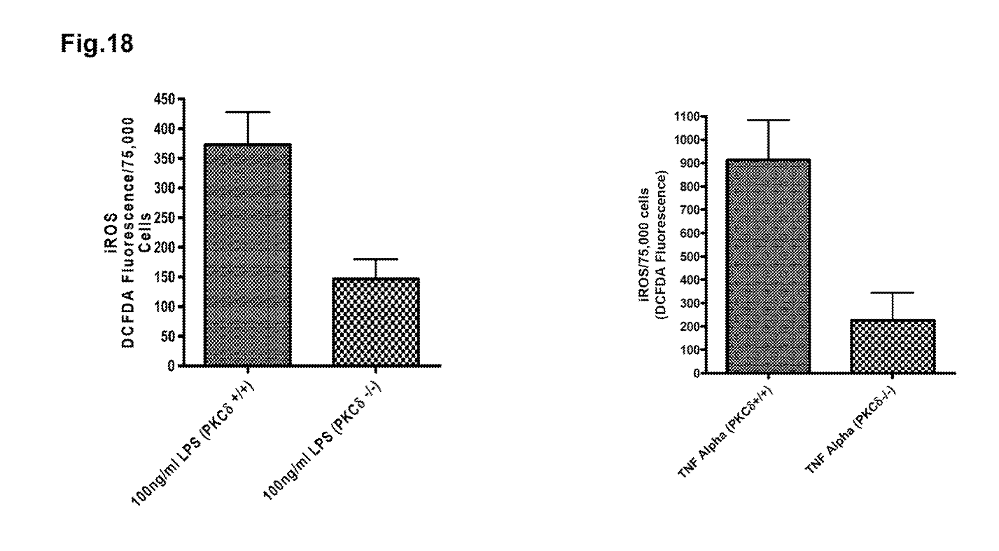

FIG. 18. Primary Microglia from PKCδ Knockout Mice Have Attenuated Superoxide generation Generation (ROS production) FIG. 18. Primary Microglia isolated from PKC-delta wild type (PKCδ +/+) and knockout (PKCδ−/−) mice were treated with LPS or TNF alpha for 24 hrs. Intracellular ROS generation was measured using the DCFH-DA dye and the increase in ROS was quantified using the control wells for background subtraction. The results show that Primary Microglia from PKCδ Knockout Mice Have Attenuated Superoxide generation Generation (ROS production).

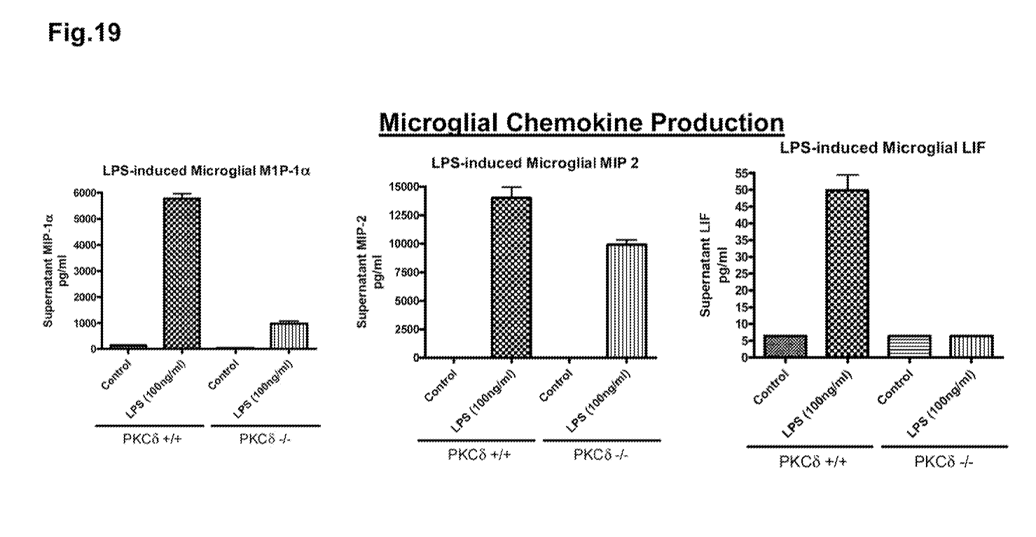

FIG. 19. Primary Microglia isolated from PKC-delta wild type (PKCδ +/+) and knockout (PKCδ−/−) mice were treated with LPS for 24 hrs. Supernatants were collected and the chemokines were quantified using the luminex immunoassay system with recombinant standards. The results show that microglia from PKC-delta Knockout (−/−) mice have reduced cytokine and chemokine production upon LPS activation.

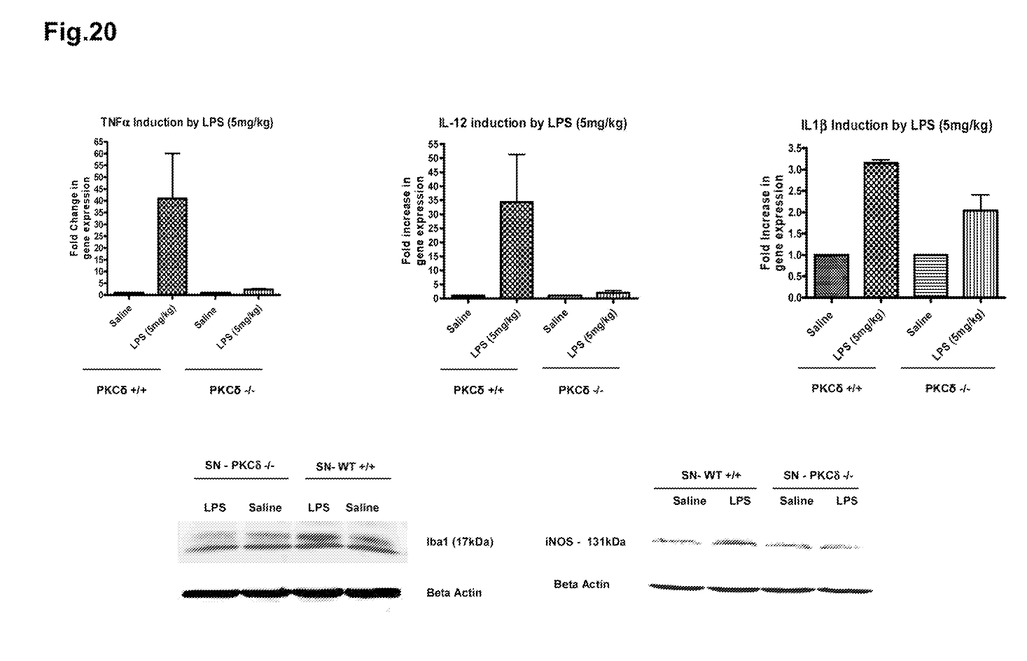

FIG. 20. PKCdelta wild type (+/+) and Knockout (−/−) mice were treated were injected with 1 dose of LPS (5 mg/kg; i.p.) and sacrificed 24 hrs later. The substantia nigra and striatum were dissected using a brain matrix. Cytokine levels in the brain tissue was determined using quantitative SYBR green real time PCR. Western blots for the microglial marker iba1 and iNOS were also used to determine microglial activation. The results show that PKCδ Knockout Mice Have Reduced Cytokine Production and Microglial Activation in the Mouse SNpc and Striatum Following Systemic LPS Challenge.

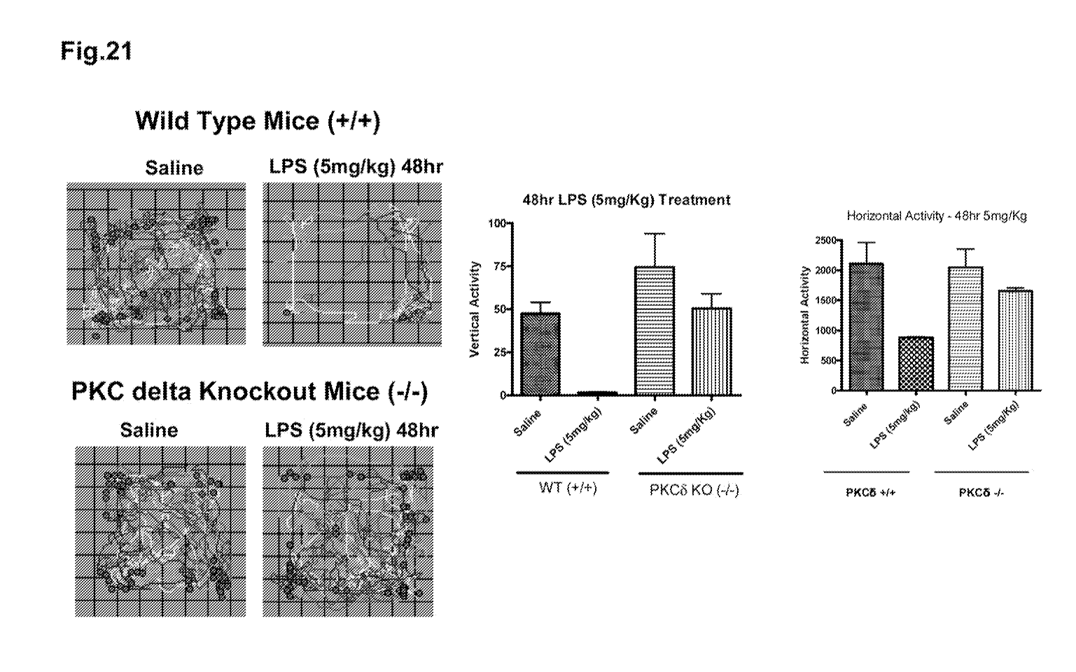

FIG. 21. PKCdelta wild type (+/+) and Knockout (−/−) mice were treated were injected with 1 dose of LPS (5 mg/kg; i.p.) and behavioral studies were performed 48 hours later using the versamax infrared analyzer. Vertical and horizontal activity amongst other parameters were measured and the data was quantified and expressed graphically. deficits. The results show that PKCδ Knockout Mice have reduced levels of serum cytokines and chemokines following systemic LPS challenge and indicate that PKCδ Knockout Mice are resistant to systemic LPS-induced sickness behavior and motor.

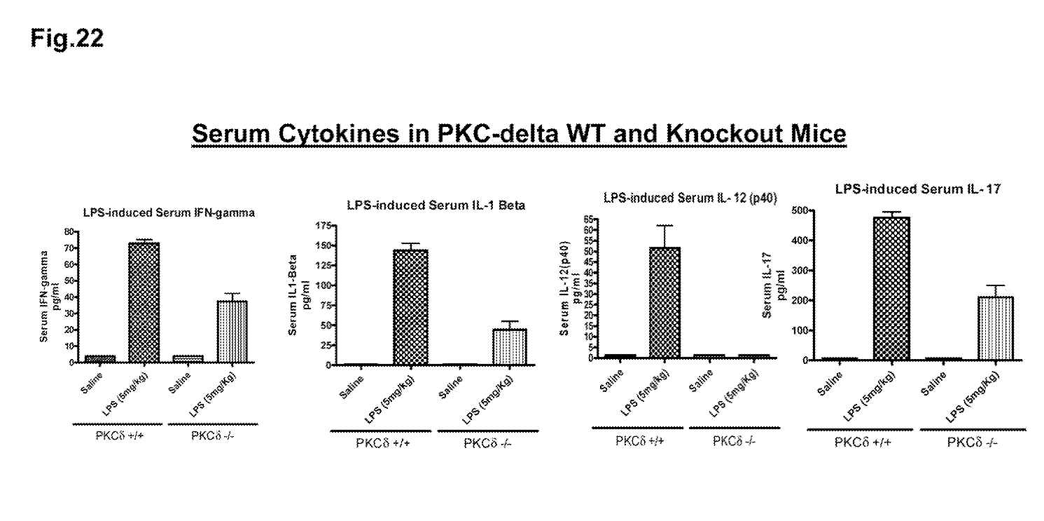

FIG. 22. PKCdelta wild type (+/+) and Knockout (−/−) mice were treated were injected with 1 dose of LPS (5 mg/kg; i.p.) and serum was collected 48 hours later by cardiac puncture. Serum cytokines and chemokines were quantified using the luminex immunoassay system. The results show that PKCδ Knockout Mice have reduced levels of serum cytokines and chemokines following systemic LPS challenge.

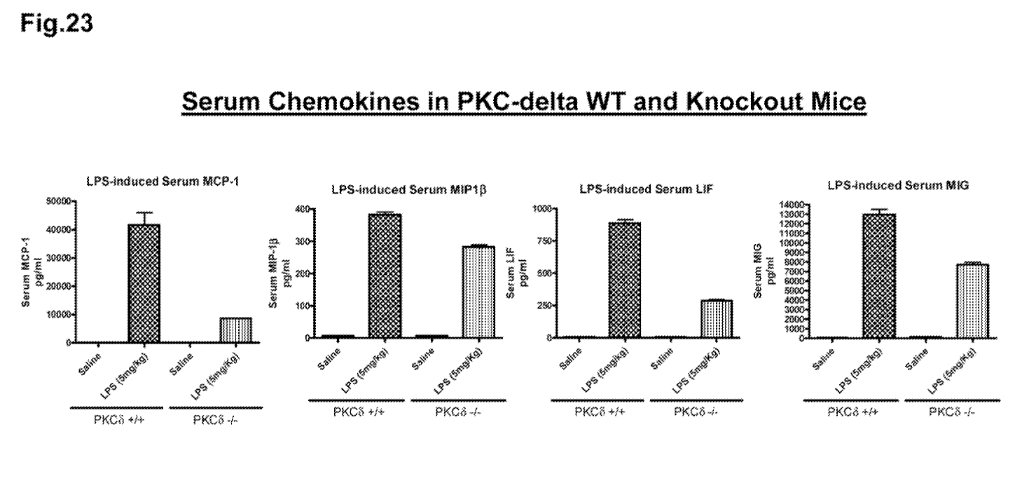

FIG. 23. PKCdelta wild type (+/+) and Knockout (−/−) mice were treated were injected with 1 dose of LPS (5 mg/kg; i.p.) and serum was collected 48 hours later by cardiac puncture. Serum cytokines and chemokines were quantified using the luminex immunoassay system. The results indicate that PKCδ Knockout Mice have reduced levels of serum cytokines and chemokines following systemic LPS challenge.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

What is claimed is:

1. A method for treating or preventing inflammation in the central nervous system of a mammal, comprising administering to a mammal in need thereof an effective amount of a PKCd inhibitor.

2. The method of claim 1, wherein the amount of PKCd inhibitor is an amount effective for decreasing inflammation in the central nervous system.

3. The method of claim 1, wherein the amount of PKCd inhibitor is an amount effective for preventing inflammation in the central nervous system.

4. The method of claim 1, wherein the amount of PKCd inhibitor is an amount effective for decreasing the release of pro-inflammatory substances from an activated microglial cell.

5. The method of claim 4 wherein the one or more pro-inflammatory substance is a cytokine, chemokine, protease, prostaglandin, leukotriene, thromboxane, neurotoxin, or a combination thereof.

6. The method of claim 5 wherein the cytokine is IL-1, IL-6, IL-8, IL-10, IL-12, IL-18, IP-10, Tumor Necrosis Factor (TNFα), cyclooxygenase-2, or Tumor Necrosis Factor (TNF-beta).

7. The method of claim 5 wherein the chemokine is IL8, Macrophage Inflammatory Protein (MIP-1α, MIP-1β), Monocyte chemotactic protein (MCP-1), Macrophage antigen complex-1 (MAC1), Stromal cell derived factor-1 (SDF-1), or Regulated upon Activation Normal T cell Expressed and Secreted (RANTES).

8. The method of claim 5 wherein the protease is cathespin B, L, and S, the matrix metalloproteinase MMP-1, MMP-2, MMP-3, or MMP-9.

9. The method of claim 5 wherein the neurotoxin is a reactive oxygen species.

10. The method of claim 9 wherein reactive oxygen species is superoxide, nitric oxide, peroxynite, or hydrogen peroxide.

11. The method of claim 1 wherein the PKCdelta inhibitor is a polynucleotide, peptide, polysaccharide, lipid, small molecule or drug.

12. The method of claim 11, wherein said PKCdelta (PKCd) inhibitor is (3-[(8-Cinnamoyl-5,7-dihydroxy-2,2-dimethyl-2H-1-benzopyran-6-yl)methyl]-2′,4′,6′-trihydroxy-5-methylaceophenone (rottlerin).

13. The method of claim 5 wherein the PKCdelta inhibitor decreases PKCd activity or expression in a microglial cell or the translocation of PKCd to the plasma membrane of the microglial cell.

14. The method of claim 1 wherein the administration of the PKCdelta inhibitor results in a decrease in the neuroinflammation in the central nervous system.

15. The method of claim 1 comprising administering the PKCdelta inhibitor to the central nervous system of the mammal, wherein the mammal has a disease, disorders or conditions associated with the release of pro-inflammatory substances from an activated microglial cell.

16. The method of claim 1 wherein the disease, disorder or condition is a neuroinflammatory disease, disorder or condition.

17. The method of claim 1, wherein the central nervous system is the brain or spinal cord.

18. The method of claim 1, wherein the PKCd inhibitor is administered orally, intravenously, intramuscularly, subcutaneously, intraorbitally, intracapsularly, intraperitoneally, intrarectally, intracisternally, intraparenchymally to the mammal.

19. The method of claim 1, wherein the PKCd inhibitor is administered prophylactically.

20. The method of claim 1, wherein the PKCd inhibitor is administered prior to inflammation of the CNS or the onset of neurodegeneration.

21. The method of claim 1, wherein the PKCdelta inhibitor is administered concurrently or sequentially with one or more an anti-inflammatory agents.

22. A method for identifying compounds that inhibit the production and/or release of pro-inflammatory substances within activated microglial cells comprising
(a) contacting microglial cells with a PKCd inhibitor, wherein the microglial cells are activated;
(b) determining the amount of one or more pro-inflammatory substances within the microglial cells or released thereby; and
(c) comparing the amount found in step (b) with an amount of pro-inflammatory substances found in the absence of the a PKCd inhibitor, wherein a decrease in the amount of pro-inflammatory substances in the presence of the PKCd inhibitor indicates the PKCd inhibitor is a compound that inhibits the production and/or release of pro-inflammatory substances.

23. The method of claim 22, wherein the one or more pro-inflammatory substance is a cytokine, chemokine, protease, prostaglandin, leukotriene, thromboxane, neurotoxin, or a combination thereof.

24. The method of claim 23, wherein the one or more pro-inflammatory substance is IL-1, IL-6, IL-8, IL-10, IL-12, IL-18, IP-10, Tumor Necrosis Factor (TNFα), cyclooxygenase-2, Tumor Necrosis Factor (TNF-beta), IL8, Macrophage Inflammatory Protein (MIP-1α, MIP-1β), Monocyte chemotactic protein (MCP-1), Macrophage antigen complex-1 (MAC1), Stromal cell derived factor-1 (SDF-1), Regulated upon Activation Normal T cell Expressed and Secreted (RANTES). cathespin B, L, and S, the matrix metalloproteinase MMP-1, MMP-2, MMP-3, or MMP-9, a superoxide, nitric oxide, peroxynite, hydrogen peroxide, NADPH oxidase, NADPH oxidase isoform, nitric oxide, or a nitric oxide synthase isoform.

25. The method of claim 22, wherein the PKCdelta inhibitor is a polynucleotide, peptide, polysaccharide, lipid, small molecule or drug.

26. The method of claim 25, wherein said PKCdelta (PKCd) inhibitor is (3-[(8-Cinnamoyl-5,7-dihydroxy-2,2-dimethyl-2H-1-benzopyran-6-yl)methyl]-2′,4′,6′-trihydroxy-5-methylaceophenone (rottlerin).

27. The method of claim 22, wherein the PKCdelta inhibitor decreases PKCd activity or expression in a microglial cell or the translocation of PKCd to the plasma membrane of the microglial cell.

28. The method of claim 22, wherein the PKCdelta inhibitor is a test compound.

29. The method of claim 22, wherein the PKCdelta inhibitor is a polynucleotide, peptide, polysaccharide, lipid, small molecule or drug.

30. A pharmaceutical composition for decreasing levels of pro-inflammatory substances in a central nervous system of a mammal in need thereof comprising an amount effective of a protein kinase C delta (PKCd) inhibitor effective for decreasing levels of pro-inflammatory substances released from activated microglial cells and a pharmaceutically acceptable carrier.

31. The pharmaceutical composition of claim 30, wherein said composition is useful in preventing neuroinflammation.

32. The pharmaceutical composition of claim 30, wherein said PKCdelta (PKCd) inhibitor is a polynucleotide, peptide, polysaccharide, lipid, small molecule or drug.

33. The pharmaceutical composition of claim 30, wherein the PKCd inhibitor is (3-[(8-Cinnamoyl-5,7-dihydroxy-2,2-dimethyl-2H-1-benzopyran-6-yl)methyl]-2′,4′,6′-trihydroxy-5-methylaceophenone.

34. The pharmaceutical composition of claim 30, wherein the PKCdelta inhibitor decreases PKCdelta activity or expression in a microglial cell or the translocation of PKCd to the plasma membrane of the microglial cell.

35. The pharmaceutical composition of claim 30, wherein said PKCd inhibitor is administered to a mammal prior, during or subsequent to neuroinflammation.

36. The pharmaceutical composition of claim 30, wherein said PKCd inhibitor is administered to a mammal prior to the onset of a neurodegenerative disease.

37. The pharmaceutical composition of claim 30, wherein the PKCdelta inhibitor is a test compound.

38. The pharmaceutical composition of claim 37, wherein the PKCdelta inhibitor is a polynucleotide, peptide, polysaccharide, lipid, small molecule or drug.

39. A method for decreasing the amount of one or more pro-inflammatory substances released from an activated microglial cell comprising contacting a microglial cell with an effective amount of a PKCdelta inhibitor.

40. The method of claim 39, wherein the amount of PKCd inhibitor is an amount effective for decreasing the release of pro-inflammatory substances from an activated microglial cell.

41. The method of claim 39, wherein contacting the microglial cell with the PKCdelta inhibitor results in a decrease in the release of pro-inflammatory substances as compared to that which would occur in the absence of the PKCdelta (PKCd) inhibitor.

42. The method of claim 39, wherein contacting the microglial cell with the PKCd inhibitor results in a decrease in the production of inflammatory substances within the microglial cell as compared to that which would occur in the absence of the PKCd inhibitor.


SEQUENCE LISTING (19 sequences)

SEQ ID NO: 1 - DNA, 29 bp, rattus norvegicus
aagattcactacatcaagaaccctgtctc

SEQ ID NO: 2 - DNA, 29 bp, rattus norvegicus
aagttcttgatgtagtgaatccctgtctc

SEQ ID NO: 3 - DNA, 29 bp, rattus norvegicus
aaggtactttgcaatcaagtacctgtctc

SEQ ID NO: 4 - DNA, 29 bp, rattus norvegicus
aatacttgattgcaaagtacccctgtctc

SEQ ID NO: 5 - DNA, 29 bp, rattus norvegicus
aacatcaggcttcacccctttcctgtctc

SEQ ID NO: 6 - DNA, 29 bp, rattus norvegicus
aaaaaggggtgaagcctgatgcctgtctc

SEQ ID NO: 7 - DNA, 29 bp, rattus norvegicus
aactgtttgtgaatttgccttcctgtctc

SEQ ID NO: 8 - DNA, 29 bp, rattus norvegicus
aaaaggcaaattcacaaacagcctgtctc

SEQ ID NO: 9 - RNA, 21 nt, Homo sapiens
aauccacuacaucaagaacuu

SEQ ID NO: 10 - RNA, 21 nt, Homo sapiens
guucuugauguaguggauuuu

SEQ ID NO: 11 - RNA, 21 nt, Homo sapiens
cugugugugaaucugcuuuuu

SEQ ID NO: 12 - RNA, 21 nt, Homo sapiens
aaagcagauucacacacaguu

SEQ ID NO: 13 - RNA, 22 nt, rattus norvegicus
gcaucuccuucaauuccuauuu

SEQ ID NO: 14 - RNA, 22 nt, rattus norvegicus
auaggaauugaaggagaugcuu

SEQ ID NO: 15 - RNA, 23 nt, rattus norvegicus
gcaguuucuacacagcaaagguu

SEQ ID NO: 16 - RNA, 23 nt, rattus norvegicus
ccuuugcuguguagaaacugcuu

SEQ ID NO: 17 - RNA, 23 nt, rattus norvegicus
gccucaccgauucaagguuuauu

SEQ ID NO: 18 - RNA, 23 nt, rattus norvegicus
uaaaccuugaaucggugaggcuu

SEQ ID NO: 19 - protein, 4 aa, Homo sapiens
DIPD